(19) United States
(12) Patent Application Publication
(10) Pub. No.: US 2010/0190004 A1
(43) Pub. Date: Jul. 29, 2010

(54) ANTIMICROBIAL LAMINATE CONSTRUCTS

(76) Inventors: Bruce L. Gibbins, Lake Oswego, OR (US); Bhalchandra M. Karandikar, Tigard, OR (US)

Correspondence Address: Ballard Spahr LLP, SUITE 1000, 999 PEACHTREE STREET, ATLANTA, GA, 30309-3915, US

(21) Appl. No.: 12/625,263

(22) Filed: Nov. 24, 2009

Related U.S. Application Data
(60) Provisional application No. 61/117,275, filed on Nov. 24, 2008.

(51) Int. Cl. A61F 13/02; B05D 5/10; B32B 15/04

(52) U.S. Cl. 428/346; 428/343; 428/457; 427/2.31; 428/355 AC; 428/355 N; 428/355 EN

(57) ABSTRACT

The present invention comprises methods for making and using antimicrobial laminate constructs comprising an antimicrobial layer and optionally, an adhesive layer. The present invention comprises methods for making medical devices, surfaces that may be in contact with medical equipment, personnel or patients, or treatment areas antimicrobial comprising, for example, applying an antimicrobial laminate construct.

### RELATED APPLICATION

This application claims benefit of U.S. Provisional Application No. 61/117,275, filed Nov. 24, 2009, which is incorporated herein by reference in its entirety.

### TECHNICAL FIELD

The present invention relates to antimicrobial laminate constructs, more particularly to methods and compositions for making antimicrobial laminate constructs and use of such laminates to render a surface antimicrobial.

### BACKGROUND OF THE INVENTION

Despite the continued development of new and more powerful antibiotics, coupled with increased stringency in hygiene, the incidences of hospital acquired infections are on the rise. Many hospital acquired infections involve antibiotic resistant strains of bacteria such MRSA and VRE which can lead to added expense in treatment costs and patient fatalities. Many hospital acquired infections result from the medical devices used in the management or treatment of patients.

The medical device industry has been actively pursuing methods for decreasing the colonization of devices by opportunistic organisms and the conduits for infection. Medical devices are generally made from materials that are biocompatible, but an unfortunate by-product of the use of biocompatible materials is that those materials are also very compatible environments for microbial colonization and growth. Organisms colonize the surfaces of medical devices to establish a critical mass of organisms and this leads to infections for the patient associated with the medical device. Very often these devices are either implanted or indwelling, and colonization by organisms creates problems for the device, the patient, and leads to changes in use of the device or the treatment regimen.

Device makers have sought ways to impart antimicrobial aspects to medical devices. The widespread use of silver as an antimicrobial in wound care products can largely be attributed to the fact that straightforward methods for coupling the materials of the wound dressing with silver have been found. This has not been possible with the wide variety of materials used in making many other types of medical devices. Technologies such as SilvaGard (AcryMed), which is an aqueous dip application process of silver nanoparticles might be suitable for many finished medical devices. Other strategies for imparting antimicrobial effect include direct incorporation of silver into the materials used to make the device. This may be useful for materials that are hydrophilic in nature or highly porous but are not suitable for devices made from metal or polymeric materials. Applying antimicrobial agents by dipping or incorporation into the material is not a viable solution for rendering antimicrobial materials that are manufactured in roll or sheet stock that may serve as the precursor of medical device components that are cut from the material. For example, roll to roll materials, or foams or paper products initially made in sheet stock would not be amenable or dipping or incorporation of antimicrobial materials due to factors such as cost to manufacture and alterations to the base material making it unusable. Additionally, such methods do not allow for easily making portions, components or particular surfaces of medical devices antimicrobial. What is needed are methods and devices that are suitable for making surfaces, such as medical device surfaces, antimicrobial.

### SUMMARY OF THE INVENTION

The present invention comprises antimicrobial laminate constructs, methods of making the constructs, methods of using the constructs for making medical devices, treatment areas, patient contact surfaces and materials antimicrobial, compositions comprising antimicrobial agent or agents used in making the constructs and methods of making the compositions. Antimicrobial herein means reduction or inhibition of microbial bioburden, colonization, or attachment by microbial organisms. A method for making a surface antimicrobial comprises applying to a surface an antimicrobial laminate construct.

An aspect of the present invention comprises a construct comprising an antimicrobial layer. An antimicrobial layer comprises one or more antimicrobial agents such as silver or other active agents. A construct may further comprise a second layer. A second layer may comprise an adhesive or other attachment compositions, or other compounds desired for the construct. When the antimicrobial layer and a second layer are in contact, a laminate construct is formed. For example, an antimicrobial laminate construct may comprise a laminate as a sheet or continuous roll comprising two layers, an antimicrobial layer and a second layer comprising an adhesive, whereby one layer comprises an antimicrobial agent, wherein one surface of the antimicrobial layer is contacting substantially all of a surface of a second layer comprising an adhesive. In use the adhesive layer contacts a surface and secures the antimicrobial layer to the surface so that the antimicrobial layer is outermost.

An example of using a laminate construct is in applying a laminate comprising at least an antimicrobial layer and an adhesive layer, in a manner like wall paper. For example, a release liner contacting the adhesive layer is removed to expose the adhesive which then contacts the surface, such as by nip-rolling onto a material such as a sheet of foam, so that the antimicrobial layer is now the outermost surface of the material, the sheet of foam. A release liner may be attached to the outer surface of the antimicrobial layer to prevent exposure to the environment and protect the layer. The foam with laminate construct attached may be cut into any desirable shape by shear cutting or die stamping, and the release liner covering the antimicrobial layer may remain in place or be removed. Oriented in this fashion the adhesive layer bonds the antimicrobial layer to the surface of the foam making the pre-made foam now antimicrobial on the side that has received the application of the laminate construct.

An antimicrobial laminate construct may comprise one or more antimicrobial layers and/or one or more second layers, such as adhesive layers, or may comprise only one of either an antimicrobial or a second layer, such as an adhesive layer. Constructs may be applied to any surface where reduction of bioburden or microbial inhibition is desired.

A method of making an antimicrobial laminate construct as a sheet or continuous roll comprises coating an antimicrobial composition on one surface of a structural element, such as a first release liner and optionally, drying the coating, forming an antimicrobial layer. A second layer comprising an adhesive composition is applied to a second structural element, such as the surface of a second release liner, which optionally may be dried, forming an adhesive layer. The outer surface of the antimicrobial layer is placed on the outer surface of the adhesive layer form an antimicrobial laminate construct having an antimicrobial layer and an adhesive layer, with release liners on the two outer surfaces.

An example of a method of making an antimicrobial laminate construct comprises coating an antimicrobial composition on one surface of a structural element, such as first release liner to form an antimicrobial layer, optionally drying the antimicrobial layer, coating an adhesive composition directly on to the surface of the antimicrobial layer opposite the surface contacting the structural element. The second coating is optionally dried, and a laminate construct is formed. A second structural element, such as a release liner may be applied to the outer surface of the adhesive layer.

An antimicrobial laminate construct of the present invention can provide an antimicrobial aspect to any surface by application of the laminate to the surface, such as by contacting an adhesive layer with the surface. A method of making a surface antimicrobial comprises contacting the outer surface of an adhesive layer of a laminate to the surface and thus providing the antimicrobial layer as the outermost layer of the surface. The method may further comprise removal of structural elements from one or more surfaces.

An antimicrobial layer may comprise an antimicrobial composition. An antimicrobial composition may comprise one or more antimicrobial agents, one or more solvents, a binder, optionally, a plasticizer, and optionally other additives. The amount of antimicrobial agents in the compositions may depend on the duration of the antimicrobial effect desired. An adhesive layer may comprise an adhesive composition comprising one or more adhesives, one or more solvents, and optionally a composition such as a binder that generally possesses good film forming property. Methods of making antimicrobial compositions and adhesive compositions are also encompassed by the present invention.

### DESCRIPTION OF FIGURES

FIG. 1 shows an exemplary laminate construct.

FIGS. 2A and B show an exemplary laminate construct and its attachment to a surface.

FIG. 3 shows an exemplary laminate construct.

FIGS. 4A and B show an exemplary laminate construct and its attachment to a surface.

### DETAILED DESCRIPTION

The present invention comprises antimicrobial laminate constructs comprising an antimicrobial layer; methods of making the constructs; methods of using the constructs for making medical devices, treatment areas, patient contact surfaces and materials antimicrobial; antimicrobial compositions comprising one or more antimicrobial agents for making an antimicrobial layer, adhesive compositions, and methods of making the compositions. As used herein antimicrobial means reduction or inhibition of microbial bioburden, colonization, growth or attachment by microbial organisms. Uses for the present invention comprise making surfaces or sites antimicrobial. Sites for insertion of medical devices into humans or animals are ideal as a portal of entry for microbes and are often colonized by bacteria or other microbes. The present invention aids in reducing the microbial growth at such sites or maintaining a site relatively free from harmful microbial growth.

An antimicrobial agent that may be used in the present invention is silver. Silver has been incorporated into wound care products and other medical devices to serve as an antimicrobial agent. Silver has emerged as a favored broad spectrum antimicrobial of choice because it is very active against bacteria and fungi in very small quantities, such as 0.1 ppm, and it is non-toxic to tissue cells at those low concentrations. Though the mechanism of action of silver is poorly understood, it is believed that it is only active as an antimicrobial in the ionic Ag⁺ form or other charged forms. It is believed to act as an oxidant that reacts readily with nucleophilic groups of many compounds found in biological organisms. The strong binding characteristics, coupled with the oxidizing effect of ionic silver means that it likely disrupts normal biological functions of bound ligands. There is a low risk of developing silver resistance by microbial strains. Additionally, silver, unlike other antimicrobial heavy metals, is seldom associated with contact sensitivity in users.

Though examples of antimicrobial compositions and layers comprising silver are taught herein, other antimicrobial agents are contemplated by the present invention. Antimicrobial composition, including but not limited to silver, may be incorporated directly into a substrate, such as a polymeric matrix foam, during the manufacture of the substrate. An antimicrobial composition may be adsorbed or absorbed by a substrate, such as a woven or nonwoven material, or a polymeric material such as a carboxymethylcellulose, or an antimicrobial composition may be plated, electroplated, spray-coated or sputter coated onto a substrate. Antimicrobial compositions and substrates may be considered pre-made antimicrobial layers and may be used in laminate constructs.

An aspect of the present invention comprises a construct comprising an antimicrobial layer. An antimicrobial layer comprises at least an antimicrobial agent such as silver or other active agents. A construct may further comprise a second layer. A second layer may comprise an adhesive or other attachment compositions. When the antimicrobial layer and a second layer are in contact, a laminate construct is formed.

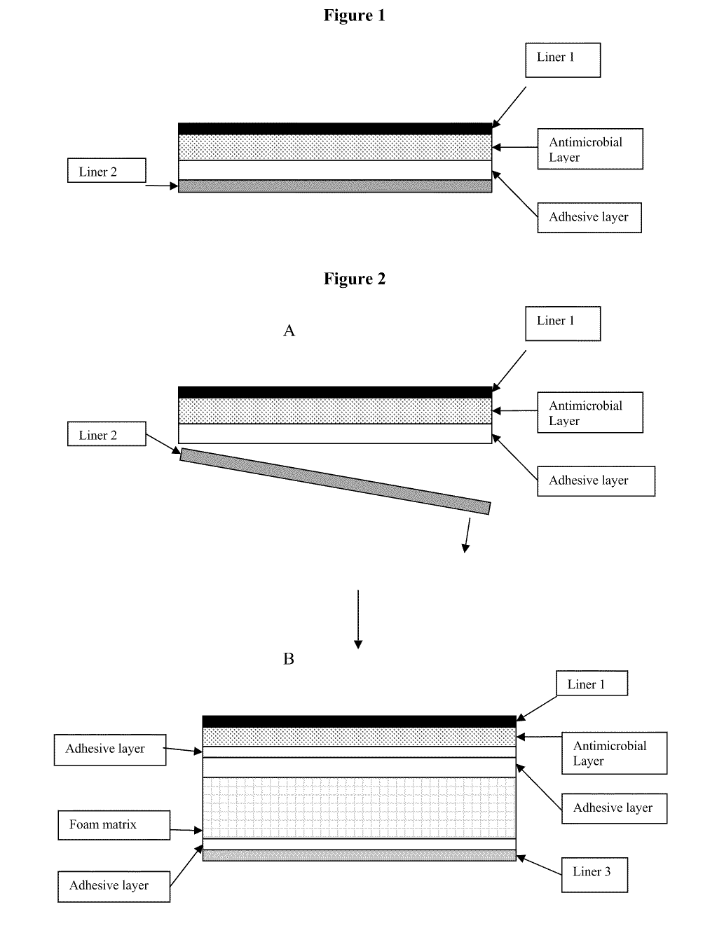

An antimicrobial layer and a second layer may be in contact so that substantially all of the surface of one side of the antimicrobial layer is contacting substantially all of the surface of one side of the second layer. For example, the laminate may be in a film or sheet or continuous film or sheet roll form with an adhesive outer surface and an antimicrobial outer surface. Alternatively, one side of the antimicrobial layer may contact substantially all or only a portion of one side of a second layer. Such arrangement of a layer or layers of a laminate construct can be determined by the use of the laminate and are within the skill of those in the art. An example of a laminate construct contemplated by the present invention is shown in FIG. 1. It comprises an antimicrobial layer and an adhesive layer sandwiched between a pair of release liners.

A laminate may comprise one layer of an antimicrobial layer and one layer of a second layer, such as an adhesive layer. A laminate may comprise may comprise multiple antimicrobial layers, which may or may not have the same antimicrobial agents, with one or more second layers, which may or may not comprise one or more adhesive layers. For example, a laminate comprises more than one antimicrobial layers with an adhesive layer contacting the entire surface of one of the outermost layers. The adhesive layer is used to attach the laminate to a surface with the outermost antimicrobial layer exposed to the environment. The outermost antimicrobial layer is exposed and releases its one or more antimicrobial agents. With use or over time, as the antimicrobial activity decreases, the outermost antimicrobial layer is removed and the next antimicrobial layer is exposed and provides renewed antimicrobial activity to the site. One or more antimicrobial layers may alternate with one or more second layers. One or more layers may have a structural element, such as a liner, between the layers. Alternatively, an antimicrobial composition may be admixed with an adhesive so that the laminate comprises one layer comprising an antimicrobial composition and an adhesive composition, and optionally one or more structural elements such as a release liner.

A laminate construct of the present invention comprises an antimicrobial layer. An antimicrobial layer is made from an antimicrobial composition, which is intended to mean that an antimicrobial layer comprises the components of an antimicrobial composition, except for those that may be removed, decreased or added in making the antimicrobial layer, such as removal of some portion or all of one or more solvents from the antimicrobial composition by drying the antimicrobial composition applied to a structural element. For example, an antimicrobial composition is applied to a structural element, such as a release liner, and some or all of the one or more solvents or other liquids in the antimicrobial composition are removed, such as by heating or drying, to form an antimicrobial layer which comprises the remaining components of the antimicrobial composition. As used herein, the terms an antimicrobial composition and an antimicrobial layer are interchangeable and their meaning and use is clear from the description. An antimicrobial composition comprises one or more antimicrobial agents. An antimicrobial composition may comprise other components such as solvents for the one or more antimicrobial agents, solvents for film-forming agents, film-forming agents, binders, plasticizers, or other components used in making an antimicrobial composition.

An antimicrobial composition may comprise an antimicrobial agent, such as those described herein or others, and an adhesive composition. For example, an adhesive such as Aeroset 1920-Z52, (Ashland Chemical Company) may be added to the antimicrobial composition. The antimicrobial composition may or may not have adhesive properties. An antimicrobial composition comprises at least one antimicrobial agent, a binder which is an agent or composition that enables the formation of a film, and a plasticizer which is an agent or composition that provides elasticity and flexibility for the antimicrobial layer.

Antimicrobial herein means reduction or inhibition of microbial bioburden, colonization, or attachment by microbial organisms. Antimicrobial agents comprise compounds, molecules and chemical elements that are antimicrobial, including but not limited to, antibiotics, antiseptics or other antimicrobial compounds, silver, silver nanoparticles, ionic silver, combinations of one or more one silver compounds, other metals such as zinc, copper, gold, platinum, and their salts or complexes, for example, zinc undecylenate, quaternary ammonium salts, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, chlorohexidine, polyhexamethylene biguanides, polyhexamethylene biguanides, triclosan, iodine, iodine-polyvinyl pyrrolidone complex, urea-peroxide complex, benzalkonium salts, quaternary ammonium compounds based on saccharinate such as Onyxide (Stepan Chemical), turmeric extract, other natural anti-infective compounds and combinations thereof. Examples of antimicrobial agents suitable for use in the present invention are agents that can be dissolved or dispersed as fine particles or be present on or in inert supports. Polymeric antimicrobial compositions are also comprised by the present invention. For example, an antimicrobial moiety may be part of a polymer. Examples of such polymer-based antimicrobials are disclosed in U.S. Pat. Nos. 5,149,524; 5,354,862; and 5,508,417 and each is incorporated herein in its entirety. Copper and zinc compounds that may be used in the present invention are listed in The Merck Index 11th Edition (1989) and known to those skilled in the art.

Silver-containing compositions and methods are disclosed herein for exemplary purposes, and are not intended to be limiting to the invention. For example, silver saccharinate (AgSacc) is taught as an antimicrobial agent but other antimicrobial agents or combinations of antimicrobial agents may be used without departing from the scope of the invention. For instance, an antimicrobial composition may comprise a fast acting antimicrobial agent, for example, clorohexidine gluconate (CHG) only or may comprise CHG and a longer term antimicrobial agent such as AgSacc, in the antimicrobial layer. Methods and compositions of the present invention comprise laminate constructs comprising silver and/or other antimicrobial agents. The antimicrobial function in the present invention may be provided by a single antimicrobial agent or by a combination of antimicrobial agents. A silver compound may be one of the antimicrobial agents. Examples of antimicrobial agents that may be used in the laminate constructs of the present invention are taught in U.S. Pat. No. 6,605,751, PCT/US2005/027261 and PCT/US2005/027260, each of which are incorporated herein in its entirety.

An antimicrobial composition may optionally comprise other additives. For instance, colorants may be added to tint the layer. Colorants may be synthetic or natural. Suitable colorants are food colors approved by FDA. Fluorescent compounds may be added to an antimicrobial composition. Fillers such as titania, natural or synthetic clays (Laponite® for example) and other fillers known to be used in cosmetic industry to provided color shades may be added. Humectants such as glycerol, urea, glycols, PEG, polyethylene glycol, and higher molecular weight analogs, may also be included in an antimicrobial composition. Plasticizers, such as those disclosed in U.S. Pat. No. 6,605,751, glycerol in water, propylene glycol and butanol may be incorporated in an antimicrobial composition. Low molecular weight polyamide resins used in the dental industry may also serve as plasticizers.

An antimicrobial compound such as a silver salt may be formed in situ in an antimicrobial composition by using the appropriate stoichiometry of combining a soluble silver salt and an anion to form a compound of a weakly soluble silver salt. Alternatively, a weakly soluble silver compound may be separately prepared and then blended with other component to form an antimicrobial composition. Silver nanoparticles compositions, aqueous or nonaqueous, as disclosed in US Patent Application Publication No. US2007/000360, which is incorporated by reference in its entirety, may be also used with other silver compounds, or antimicrobial agents in an antimicrobial composition.

The present invention comprises antimicrobial compositions in which a range of concentrations of one or more antimicrobial agents such as silver, are used for the antimicrobial layer in laminate constructs. For example, a device having a one-time use, or a disposable device, may not require a high concentration of one or more antimicrobial agents in the antimicrobial layer of the construct, whereas long term use of a device, such as an indwelling IV access device that may be used for 3 to 7 days, may need an increased amount of the antimicrobial agent or agents in the antimicrobial layer. For example, a silver content in a laminate construct may range from 0.1 ppm to 100,000 ppm, from 0.1 ppm to 75,000 ppm, from 0.1 ppm to 50,000 ppm, 0.1 ppm to 25,000 ppm, from 0.1 ppm to 10,000 ppm, from 0.1 ppm to 5000 ppm, from 0.1 ppm to 1000 ppm, from 0.1 ppm to 500 ppm, from 0.1 ppm to 250 ppm, from 0.1 ppm to 100 ppm, from 100 ppm to 100,000 ppm, from 500 ppm to 100,000 ppm, from 800 ppm to 100,000 ppm, from 1,000 ppm to 100,000 ppm, from 5,000 ppm to 100,000 ppm, from 10,000 ppm to 100,000 ppm, from 20,000 ppm to 100,000 ppm, from 30,000 ppm to 100,000 ppm, from 40,000 ppm to 100,000 ppm. Amounts of other antimicrobial agents may range from 0.1 ppm to 50,000 ppm, in similar ranges as described. A medical device having an attached laminate construct that provides antimicrobial efficacy and yet is biocompatible, that does not irritate or stain the surrounding area or patient, is contemplated by the present invention.

An antimicrobial composition may further comprise a dispersion medium which may or may not be a solvent for the antimicrobial agent. The composition may be made of a single dispersion medium or a mixture of dispersion media. Examples of dispersion medium include, but are not limited to, water, lower alkyl alcohols (C1 to C8), branched alkyl alcohols (C1 to C8), acetone and higher ketones (MEK), mono substituted glycol ethers, acetates, lactates or formates of lower alkyl alcohols (C1 to C8), tetrahydrofuran (THF), NMP and acetonitrile.

An antimicrobial composition may comprise a binder which may be a single compound or a mixture of compounds. A binder as used herein is a compound, molecule or composition that enables the formation of a film. A binder may be a natural or synthetic polymer and may be soluble in the dispersion medium and may be inert relative to the antimicrobial agent. Binders may be low Tg (glass transition temperature) polymers or resins. Examples of binders include, but are not limited to, cellulose ether derivatives (hydroxyl alkyl cellulose with C1 to C3 alkyl groups, hydroxylpropyl methyl cellulose, methyl cellulose, ethyl cellulose, carboxy methyl cellulose), propylene alginate, polyvinyl alcohol, PVP (polyvinylpyrrolidone), polyurethanes, polyacrylates, polyacrylamides, polylactates, and combinations thereof. A binder possesses good film forming properties. Binders may also be referred to as a film-forming composition or polymer. Polymers that yield films that are flexible, elastic (to longitudinal force or bending force) and strong are contemplated by the present invention. While some of the illustrative examples disclosed use nonaqueous solvents, this should be not be construed as limiting to the invention. Both antimicrobial compositions and adhesive compositions may be entirely water based.

An example of an antimicrobial composition of the present invention comprises an antimicrobial agent and a binder. Additives such as fluorescent compounds and/or plasticizers may be added to the composition.

An antimicrobial composition may be viscous for ease in slot coating or pattern coating on a structural element such as a liner, or a woven or nonwoven material. For example, with a liner, a dispersion medium in the antimicrobial composition aids in wetting the release liner and if the dispersion medium is nonaqueous, the dispersion medium may contain a small amount of water. Methods for removing some or all of one or more solvent(s) or liquid(s) from an antimicrobial composition that has been applied to a structural element or to a second layer or to another antimicrobial layer to form an antimicrobial layer are known in the industry. Thermal heating, such as in an oven, microwave exposure, and IR (infrared) lamps are methods known in the art for removing solvents. Air-drying is also contemplated by the present invention.

The components of an antimicrobial composition may contribute to the attributes of the antimicrobial layer made from the antimicrobial composition. For example, an antimicrobial layer may be flexible, elastic to some degree in linear direction and can stretch without breaking under bending forces. The antimicrobial layer may be permeable to moisture or air, or impermeable to moisture or air, or have a very high moisture or air permeability. The antimicrobial agents of an antimicrobial layer may be agents that resist light-induced or heat-induced discoloration. An aspect of the invention may comprise antimicrobial agents, that when incorporated in the antimicrobial layer, are not affected by known sterilization methods, such as steam sterilization, ethylene oxide or gamma irradiation.

A laminate construct of the present invention may comprise a second layer. An example of a second layer is an adhesive layer. Other examples of a second layer are components for adhering or temporarily contacting an antimicrobial layer to a surface, including but not limited to double-sided tape, sticky-backed tape, or materials that provide an electrostatic cling function. An adhesive layer is made from an adhesive composition, which is intended to mean that an adhesive layer comprises the components of an adhesive composition, except for those components that may be removed, decreased or added in making the adhesive layer, such as removal of some portion or all of one or more solvents or liquids from the adhesive composition by drying the adhesive composition applied to a structural element.

For example, an adhesive composition is applied to a structural element, such as a release liner, and some or all of the one or more solvents or other liquids in the adhesive composition are removed, such as by heating or drying, to form an adhesive layer which comprises the remaining components of the adhesive composition. As used herein, the terms an adhesive composition and an adhesive layer are interchangeable and their meaning and use is clear from the description. An adhesive composition may comprise an adhesive and a solvent.

An adhesive layer of the laminate construct may comprise any type of adhesive such as a pressure sensitive adhesive, a permanent adhesive, adhesives that cure with time, light-activated adhesives that cure with electromagnetic energy such as UV or visible light, or heat-activated adhesives. Various types of adhesives that can be used in the adhesive layer of the laminate construct are known to those ordinarily skilled in the art in the coating and packaging industry. An example of a laminate construct of the present invention comprises a pressure sensitive adhesive as the adhesive layer. An adhesive composition may comprise one or more types of adhesives.

Where the antimicrobial layer and the adhesive layer are separate layers, it is contemplated that the adhesive layer does not interact with the antimicrobial layer, such as to degrade or alter the performance of the antimicrobial layer. By interaction, it is meant that the adhesive layer not cause discoloration or adverse chemical reaction to alter the function of the antimicrobial agents or not diffuse into the antimicrobial agent layer to provide an adhesive aspect within the antimicrobial layer. For example, using a binder polymer in an antimicrobial layer that does not dissolve in solvent used in an adjacent adhesive layer may prevent interaction between the two layers. Where a laminate construct comprises a separate adhesive layer and a separate antimicrobial layer, it is intended that there is no migration of adhesive into the antimicrobial layer nor movement of the antimicrobial agent into the adhesive layer. The layers are partitioned from each other, for example, by the binders used in each layer and/or the solvents used in each layer.

Adhesives used in an adhesive layer comprise pressure sensitive adhesives, which are known to those skilled in the art. A pressure sensitive adhesive may be made from polyurethane, silicone polymer, or other synthetic polymer-based, and may or may not be cross-linked. An adhesive may be natural polymer, for example, casein. The present invention contemplates adhesives that are biocompatible and inert with respect to the antimicrobial agents. For example, adhesives useful in the present invention include, but are not limited to, acrylic pressure sensitive adhesives, such as those sold commercially as DUROTAK® brand by National Starch Company; polyisobutylenes, such as those disclosed in U.S. Pat. No. 5,508,038, which is incorporated by reference in its entirety; polyacrylate based such as those of Aroset® brand adhesive from Ashland Chemical Company; stryrenic-based pressure sensitive adhesives, and BIO-PSA® brand silicone pressure sensitive adhesive (Dow Chemical Company).

An adhesive composition may optionally comprise additives. For instance, colorants may be added to tint the layer. Colorants may be synthetic or natural. Suitable colorants are food colors approved by FDA. Fluorescent compounds may be added to an adhesive composition. Fillers such as titania, natural or synthetic clays (Laponite® for example) and other fillers known to be used in cosmetic industry to provided color shades may be added. Humectants such as glycerol, urea, glycols (PEG, polyethylene glycol, and higher molecular weight analogs) may also be included in the adhesive composition. Plasticizers, such as those disclosed in U.S. Pat. No. 6,605,751, glycerol in water, propylene glycol and butanol may also be incorporated into an adhesive composition. Low molecular weight polyamide resins used in the dental industry may also serve as plasticizers.

Methods for removing the solvent(s) from an adhesive composition that has been applied to a structural element or to an antimicrobial layer, or to another adhesive or second layer, to form an adhesive layer may be those known in the industry. Thermal heating, such as an oven, microwave exposure, and IR lamps are methods known in the art for removing solvents. Air-drying is also contemplated by the present invention.

An aspect of the invention comprises a combined laminate construct made from a combination of an antimicrobial layer and a second layer to form a single layer laminate construct. For example, an antimicrobial composition and an adhesive composition are combined and mixed, and then applied to a structural element, for example a release liner, to form a combined antimicrobial and adhesive layer that has adhesive properties. The combined antimicrobial and adhesive layer may be treated to remove some or all of one or more solvents. A second release liner may be applied to the side of the laminate opposite the first release liner.

A laminate construct of the present invention may comprise a structural element. The structural element may be the structure onto which a layer, such as an antimicrobial layer or an adhesive layer, is formed. A structural element may serve to protect one or both layers from exposure to the environment. A structural element may be a permanent component of a laminate construct, such as when an antimicrobial layer is formed on a woven or nonwoven material, or a structural element may be a removable element, such as a release liner.

A structural element may be inert to an antimicrobial layer and/or to an adhesive layer. A structural element, such as a liner, may be paper, a plastic polymer or composite of paper and plastic. Silicone-based liners are well known in the art. For example, 3M ScotchPak™ brand liners may be used. For example, polyester (PET) base films with a heat-sealable polyolefin layer, which may contain a ceramic oxide coating (AlOx) are contemplated for use as a structural element or liner. The use of both paper-based and plastic-based liners are contemplated by the present invention. Paper-based liners and plastic liners come in variety of weights (# number), colors, thicknesses. A liner may be coated with silicone release material or other release materials to impart varying degrees of release rates or properties. Types of paper/plastic composite liners may also be used and are known in the art. Liners where the silicone release coating is not derived from tin based curing chemistry are contemplated, as tin is not considered GRAS. Further, the release coatings on the liners are not limited to silicone. Other materials such as fluorosilicones, or PTFE may also be suitable. For example, the thickness of a liner may range from 0.5 mils to 10 mils or higher. Liners without the release coatings may be used and still achieve the desired release performance in the laminate constructs. The choice of liner material may be based on release rate or force needed to remove the liner, or on the needs of downstream manufacturing requirements, such as ability to use die cutting or stamping operations.

An aspect of the invention comprises differential release of the liner from a layer in a laminate construct having at least two liners. By differential release, it is meant that under a specific peeling force, one liner will release and the other liner will not. In physical terms, it means the force required to release or peel one liner from one side of the construct is different from the force required to peel or release the second liner from the opposite side of the construct. In an example of a laminate construct where the antimicrobial layer is in intimate contact with the adhesive layer to form a laminate construct having an antimicrobial side and on the opposite side, an adhesive side, such as FIG. 1, it is desirable that for the liner in contact with the adhesive layer, the force required to remove it is equal or less than the force needed to remove the liner in contact with the antimicrobial layer. This aspect of differential forces is useful in mechanized operations where one of the liners remains in contact with a layer, and a second liner is removed in the operation.

In aspects, Fadhesive=Fantimicrobial. In aspects, Fadhesive<Fantimicrobial where Fadhesive is the peel force required to remove liner on adhesive side and Fantimicrobial is the force required to remove the liner contacting the antimicrobial layer. In aspects, Fantimicrobial is at least 1.5 times>Fadhesive, Fantimicrobial is at least 5 times>Fadhesive, Fantimicrobial is at least 10 times>Fadhesive. Generally, this inequality holds for the laminate construct from the time it is made to the time it is applied to a surface i.e. the ratio (Fantimicrobial/Fadhesive) should not vary over the life time of the laminate or until it gets applied to the surface. To achieve this force difference, liners typically may be coated with different release agents or materials that provide the differential release. Liners may be selected that are made from materials that have different release rates to achieve the differential release rates between at least two liners. This aids operations that apply the laminate to surfaces, so that the operations are able to first expose the adhesive layer which is then bonded to the surface that is intended to be made antimicrobial, and afterwards remove the liner covering the antimicrobial side.

The present invention comprises methods of making a laminate construct. A method of making a laminate construct composition comprises (i) applying an antimicrobial composition comprising at least one antimicrobial agent, for example, silver saccharinate (AgSacc), a dispersing liquid medium and a soluble binder on a structural element such as a protective release liner, liner 1, forming layer on the liner by coating substantially all or a portion of the liner, (ii) drying the antimicrobial composition to remove the liquid, forming an antimicrobial layer, (iii) applying a second layer to the dry antimicrobial layer, wherein the second layer comprises an adhesive composition comprising an adhesive, for example, a pressure sensitive adhesive (PSA), solvent and optionally other additives, such as a colorant, forming a coating, (iv) drying the adhesive composition to remove excess solvent forming an adhesive layer, and (v) covering the adhesive layer with a protective release liner, liner 2. A pressure sensitive adhesive is an adhesive that binds to a surface under application of pressure only and does not require activation by heat, light or solvent. A laminate construct may be manufactured in no particular order such that an adhesive layer or an antimicrobial layer may be formed first, with the other layer formed second.

A laminate construct may be provided as a discrete material that is conveniently sized and provided in individual units, or as a continuous material provided on a roll and may be used when needed to provide an antimicrobial aspect to any substrate or surface. The present invention contemplates no particular order of the formation of the layers, as in whether the adhesive layer is formed first and an antimicrobial layer is added to it, or the antimicrobial layer is formed first and the adhesive is added to it.

A method of making a laminate construct of the present invention, comprises (i) applying an antimicrobial composition comprising at least one antimicrobial agent, for example, silver saccharinate (AgSacc), and a soluble binder to a structural element such as a protective release liner, liner 1, forming layer or coating on the liner by coating substantially all or a portion of one surface of one side of the liner, (ii) drying the antimicrobial composition to form an antimicrobial layer, (iii) applying an adhesive composition comprising an adhesive, for example, a pressure sensitive adhesive, and optionally other additives, such as a colorant, to a second structural element such as a protective release liner, liner 2, forming layer on the liner by coating substantially all or a portion of the surface of one side of the liner, (iv) drying the adhesive composition to form an adhesive layer, and (v) contacting the outer surface, the surface opposite the liner, of the antimicrobial layer with the outer surface, the surface opposite the liner, of the adhesive layer, to form a laminate construct. The two surfaces may be contacted using nip rollers to exert pressure to form the laminate construct. A method of making the laminate constructs may be selected depending on the type of manufacturing equipment used and the intended use of the laminate construct.

An aspect of the invention comprises using a single structural element in making a continuous roll laminate construct. For example, only one liner is used. Optionally, when using a single liner, the two surfaces of the liner itself have different release properties. On one surface of the liner, the antimicrobial composition is applied and an antimicrobial layer is formed. The force required to release the antimicrobial layer from this surface is Fantimicrobial. An adhesive composition is applied over the antimicrobial layer and an adhesive layer is formed, and a laminate construct is made. The laminate construct is then wound into a continuous roll. When in the roll, the adhesive layer comes in contact with the side of the liner than was not coated with the antimicrobial composition. The force required to release the adhesive from the liner is Fadhesive and this force is much less than Fantimicrobial. As a result, when the laminate construct is ready to be used, one peels off the liner to expose the adhesive side first. The exposed adhesive side can be applied to any surface. After it is applied, the liner material is then peeled off to expose the antimicrobial side which is now on the outer side of the surface. It is contemplated that the ratio Fantimicrobial/Fadhesive is greater than 1 so that the liner can be released reliably from the construct. Such liners are known to those with skill in the tape industry.

A method of making a laminate construct of the present invention comprises making a laminate construct having one layer and optionally one or two structural elements. For example, an antimicrobial layer can be formed on one structural element, such as a release liner, and a second structural element, such as a second release liner, can be applied to the antimicrobial layer surface. The two liner materials sandwich an antimicrobial layer between them. The release liners may have the same or different release characteristics. A different release characteristic may be due to the type of the release coatings found on the release liners, or to the absence of a release coating on one or both of the liners. This differential release of release liners may release in such a way that one liner requires much less force compared to the other even if the liners are in contact with the same antimicrobial layer.

For example, in making such a laminate construct, an antimicrobial composition is applied on one surface of a release liner, an antimicrobial layer is formed, and a second release liner is applied to the outer surface of the antimicrobial layer, for example by passing the antimicrobial layer with the second release liner applied between a pair of nip rollers before rolling or winding the construct on a core for storage. If there is a need to remove one of the liners before the other liner, the liners may be colored or printed with instructions. In use, an antimicrobial layer only laminate construct may be applied to a surface and held in place by tape, gauze or other known methods for stabilizing material to a surface or patient. For example, one liner is removed, and the antimicrobial layer is contacted to one side of a double sided tape, the other side of the tape contacts the surface and bonds to the surface and the liner on the antimicrobial layer is removed to expose the antimicrobial layer to the environment.

In use, it is contemplated that when removing the liner contacting the antimicrobial layer, that no portion of the layer should bind to the liner material and leave a gap in the antimicrobial surface. This is also true for the adhesive layer, but gaps in adhesive may not be as detrimental to the use of the construct as gaps in antimicrobial presence or function. One way to ensure a uniform surface is provided by the antimicrobial layer is to add a colorant or fluorescent dye to the antimicrobial layer. A fluorescent dye is ordinarily not visible to humans, and would not be visible when applied to the surface. But under ultraviolet light exposure, the fluorescent compounds will fluorescence. Any missing area of antimicrobial layer would be detected as dark region.

Referring to the FIGS. 2A and 2B, the construction of a Huber needle IV access device with an antimicrobial foam cushion is shown. The liner 2 of the laminate construct may be removed to expose the adhesive layer of the laminate, which is then applied to the EVA foam cushion. Liner 1 may be removed at a later time, exposing the antimicrobial layer, thus providing an antimicrobial aspect to one surface of the foam cushion. Another surface of the foam might be treated in the same way, by application of the adhesive layer of the laminate construct and providing an antimicrobial aspect to that surface, or the foam surface may be provided with an adhesive only layer that would then allow the foam to be secured to yet another surface. An adhesive only construct may be made by applying a coating to a liner comprising a pressure sensitive adhesive, PSA, solvent and optionally other additives, such as a colorant, and forming an adhesive layer. FIG. 2 A shows removal of the liner on the adhesive layer. FIG. 2B shows an antimicrobial laminate construct attached to a surface, such as foam, and an adhesive only construct attached to the opposite side of the surface.

In the case of the Huber needle with cushion device, liner 3 is removed from the adhesive only layer and the foam construct is attached to the needle device to provide a cushion or pad. The release liner covering the antimicrobial layer is removed prior to putting the antimicrobial layer into service, for example, such as when attaching the device to the patient's body whereby the foam is provided between the needle and the patient's skin. Optionally, it may be removed prior to placing the device in packages and then appropriately sterilized. To prevent confusion between the several release liners that may be present with a laminate construct, the release liner of the antimicrobial layer may be colored, shaped, have writing on it or in some way differ from a liner used on an adhesive layer.

When in packaged form, the Huber needle IV access device with antimicrobial layer attached to the foam, ordinarily it is difficult to distinguish it from a device without an antimicrobial laminate construct. To overcome this deficiency, the present invention provides laminate constructs with a tint or colorant in one or more layers, such as in the antimicrobial layer. Alternatively, the tint or colorant can be an additive that is added to an adhesive layer instead of an antimicrobial layer, or can be added to all of the layers of the laminate construct.

A method of making a laminate construct comprises applying an antimicrobial composition to a structural element that is a fabric, such as a woven or nonwoven material. The antimicrobial composition may be applied to the fabric by any method, such as dipping or spraying, and the fabric may be impregnated with the antimicrobial composition, coated with the antimicrobial composition, and all or a portion of the fabric may be contacted by an antimicrobial composition. Alternatively, a fabric or structural support may be supplied that is antimicrobial. The fabric may be contacted by an antimicrobial agent, for example, one of those listed herein, or other antimicrobial compounds, elements or molecules, but is not contacted by an antimicrobial composition of the present invention. An antimicrobial aspect may be provided to a woven or nonwoven structural element by applying an antimicrobial layer on one side of the structural element and an adhesive layer on the opposite side.

A method of making a laminate construct comprises applying an antimicrobial composition to a structural element that is a fabric, such as a woven or non-woven fabric so that the fabric is impregnated with the antimicrobial agent. The fabric may be dipped or sprayed with an antimicrobial composition to impregnate it and is then contacted with a liner on one surface for protection. An adhesive layer may be applied directly or may be provided by contacting the fabric antimicrobial layer with an adhesive layer provided on a structural element such as a liner to complete the laminate construct. A laminate construct may comprise an antimicrobial layer applied to a fabric structural element in which both outer surfaces of the antimicrobial layer are covered by release liners.

An aspect of the present invention is that a laminate construct can be die cut to any shape and size. This allows for a laminate construct to be sized to fit any surface that is intended to be made antimicrobial. This permits effective utilization of the material, reduces waste and gives the laminate construct a competitive edge. A laminate construct can be made in the form of continuous sheet rolls of fixed dimensions that are wound on cores and would be available in standard sizes. A laminate construct can be supplied in single individual sheets that can be of any size and can be easily cut by users, such as healthcare providers.

Depending on the intended use, a laminate construct can be provided with certain characteristics. For example, laminates of a continuous roll form may be uniform with no breaks or openings in the construct layers, to provide a continuous antimicrobial surface once applied. A discontinuous antimicrobial layer could be made, for example with perforation created by forming the antimicrobial layer and/or the adhesive layer on structural elements with gravure or screen printing so that fluid or other media can move freely through the openings in the laminate construct formed. The perforations may be small (1 mm dia or less) or large (>20 mm) or any range between and depend on the end use. The perforation may be of any shape, for example, round, rectangle, square, polygon, slit or may be a random shape.

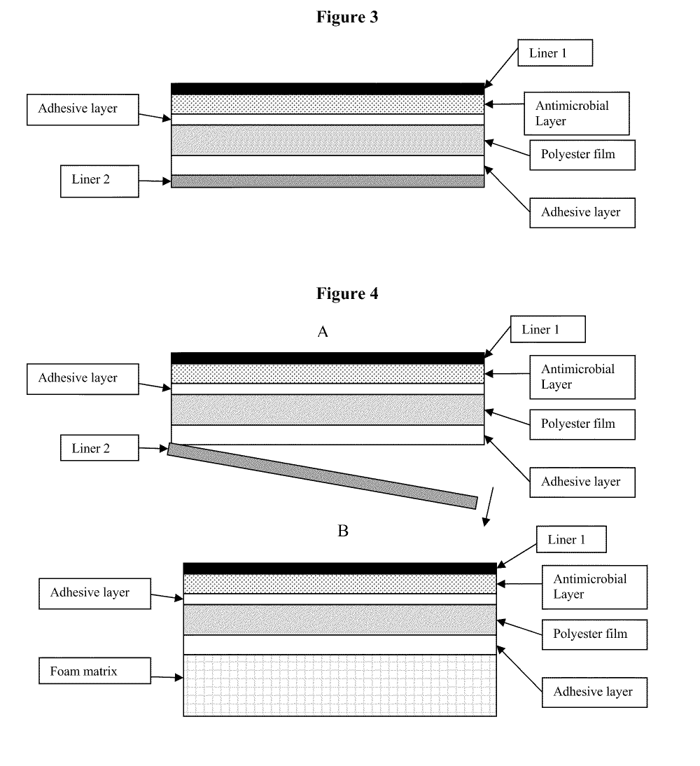

An antimicrobial layer and/or an adhesive layer may be applied so that a continuous layer is formed or the antimicrobial layer and/or an adhesive layer is formed in a pattern, such as a dot pattern. A method of making a laminate construct of the present invention may comprise forming an antimicrobial layer in an open pattern or a silk screened pattern on a structural element which is then over-coated with adhesive layer so that the adhesive layer is only formed where the antimicrobial layer is found on the structural element. When this laminate construct is transferred to the surface of a substrate such as an open cell polyurethane foam, the antimicrobial laminate construct is discontinuous and allows fluids to pass through the open areas, for example, into the foam. In the laminate constructs shown in FIGS. 1 to 4, the laminates may be continuous or may be perforated.

The laminate constructs can be applied to unlimited surface types, for example, a metal or alloy, ceramic, polymeric, plastic, glass or foam or any combination thereof. The surface contour can be flat, spherical, cylindrical or any other type of contour combination. For example, an antimicrobial laminate construct may be applied to a surface of a medical device, materials used in treatment of humans or animals, or applied to treatment areas where reduction of bioburden or inhibition of microbial growth would be advantageous, such as operating rooms, examination rooms, hospital rooms, surgical drapes or curtains, shower curtains, handle bars on doors in hospitals, flush handles on toilets, turn knobs on bathroom paper towel dispensers, toilet seats, door knobs, gurneys, cribs, beds, nasal inserts, prosthetics, walkers, canes for elderly, hospital beds and auxiliary equipment, mattresses or other surfaces contacted by medical or hospital personnel, equipment, push buttons on elevators, bathroom dryers or patients (ID tags). Non-medical applications may include lining the HVAC conduits or piping to prevent mold growth. Various examples of the end uses of the laminate constructs provided here are for illustrative purposes only and should not be construed as limiting. For example, the application of a laminate construct to a surface on an article is carried out under pressure to bond the adhesive to the underlying surface. No special efforts or procedure are needed other than ensuring the surface is ordinarily pre-cleaned to remove dirt and any residue that may hinder adhesion.

A method of making a surface antimicrobial comprises (a) providing a laminate construct and (b) attaching the laminate construct to the surface so that an antimicrobial layer is outermost. A laminate construct can be made and stored until ready to apply to a surface, such as a foam.

A device of the present invention may comprise an antimicrobial laminate construct-foam, which may be made by removing liner 2 from a laminate construct such as that shown in FIG. 1A, to expose an adhesive layer. The adhesive layer contacted to an absorbent foam, and then the antimicrobial layer is exposed by removing liner 1. The antimicrobial laminate construct bearing foam may comprise other layers such as covering the foam with a woven layer such as Tegaderm™ to create a wound care bandage that would provide antimicrobial agents to a wound, absorb exudate and not be attached to the newly formed skin.

FIG. 4A shows removal of the liner to expose an adhesive layer. FIG. 4B the adhesive side contacted with the foam surface to transfer the antimicrobial laminate onto the foam. The foam can be in the form of a roll or a sheet. It can be cut to size and then attached to a device via an adhesive to provide cushioning action.

A surface can be rendered antimicrobial multiple times by simply removing the antimicrobial agent depleted laminate construct and re-applying a fresh laminate construct or alternatively, applying a fresh laminate construct over the depleted one. For example, removal may be possible when a pressure sensitive adhesive was used in the adhesive layer. Adhesives can be removed by solvents or by abrading action following the application of mild heat. This aspect provides an advantage over other antimicrobial products (e.g. push buttons on equipments) wherein the antimicrobial agent is compounded into the base material, since once the antimicrobial in the surface is depleted, the product must be discarded or considered no longer antimicrobial, which adds costs to use of that device and loss of functionality.

Examples of devices that may have an antimicrobial incorporated to provide antimicrobial properties include devices comprising open or closed cell foams. The type of polymer used to make synthetic foam leads to finished products that have a variety of different properties. For example, polyurethane polymers form open cell foams that have a high fluid absorption capability. Fluids can readily enter the body of the foam matrix upon contact. Antimicrobial agents such as silver can be incorporated into such foams by blending the silver agent directly into the polymer mixture before the foaming step. The silver deposits throughout the matrix during the manufacturing of the foam. This type of foam may be used where absorption of fluids is a functional criterion of the device specifications. Ethylene vinyl acetate (EVA) polymers can be used to produce closed cell foams. EVA foams do not absorb fluid and are difficult to wet with an aqueous fluid. An EVA foam may be used in areas where absorption of fluid is not a functionality objective. For example, EVA foam may be used as a cushion between a continuous wear device and a patient's skin, such as in conjunction with a Huber needle used for continuous vascular port access. EVA foams are more comfortable and withstand sterilization processes better than polyurethane (PU) foams. A limitation in the use of EVA foam is that blending an antimicrobial into the polymer matrix is of little practical value since the antimicrobial would be trapped in the matrix and unavailable to control bioburden around the contact point.

In the use of a Huber needle, the patient's skin is breached by the needle and is left in place as an indwelling percutaneous device. With long time periods of use of the Huber needle, and despite the use of good hygiene practices, there is a continuous risk of infection initiating along the puncture tract. An added complication is the potential colonization of the EVA cushion by skin bacteria, which increases the microbial bioburden at the portal of access. To lessen the infection risk, foams that are poorly moisture absorbing are desired, such as closed cell foams. Therefore, there is a need for such foams to have antimicrobial properties in addition to low moisture absorption. A laminate construct can provide an antimicrobial aspect to such foams.

One method of applying antimicrobial functionality to non-moisture absorbing foam involves adding directly over the foam surface a coating of an antimicrobial silver agent. Such foam made from EVA polymer is used as a cushion element in Huber needle devices to help relieve the effect of pressure while the device is in contact with patient's arm or leg. In one example, an antimicrobial agent, silver saccharinate (hereafter referred to as AgSacc), was applied directly as a layer on the foam material using a Meyer Rod draw down technique. Although application of the antimicrobial composition directly to the foam matrix worked reasonably well under controlled laboratory conditions, alternative methods of providing an antimicrobial aspect to a device, such as foam, may be desired for full scale manufacturing. Limitations to direct coating of an antimicrobial agent include the non-uniformity of the antimicrobial coat on the uneven surfaces of materials like EVA foam sheets; the strength of adherence or bonding between the coating and the substrate; aspects related to the solvent such as in removing the solvent and control of solvent vapors, and the difficulty of applying and retaining a protective liner on the antimicrobial layer to prevent damage during storage and handling. These problems can be solved by the application of an antimicrobial laminate construct to a surface, such as an EVA foam cushion. A method of providing an antimicrobial aspect to a foam or a device, comprises attaching a laminate construct to the surface, such as a foam substrate.

The present invention comprises application and use of an antimicrobial laminate construct to render the surface of medical and non-medical devices and other surfaces antimicrobial. A medical device contemplated by the present invention comprises an antimicrobial nasal insert comprising compression molded polyethylene foam with an antimicrobial laminate construct applied to the surface. Prior to insertion into the nasal cavity, the release liner on the antimicrobial layer is removed and the device is fitted inside the nose. Such a device may be used in patients, for example in hospitals, to lower the presence of antibiotic resistant Staphylococcus aureus (MRSA). Laminate constructs of the present invention may be applied to surfaces such as to walls as wall paper. Such wall papers may be used in operating or ICU rooms in the hospitals. Bandages comprising adhesive portions around an antimicrobial pad are also contemplated by the present invention. Most medical devices or applications involving use of a foam may be made antimicrobial with the use of a laminate construct of the present invention. For example, the laminate constructs may be used to add antimicrobial function to foam tapes made by 3M® e.g. 3M 970 series foam tapes based on different polymer types.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Preparation of Silver Laminate Construct and Application to Foam

Preparation of silver saccharinate slurry: In two 50 ml conical polypropylene tubes (Falcon brand), sodium saccharinate (15 ml, 0.125M) and silver nitrate (15 ml, 0.1M) were mixed to form silver saccharinate precipitate. The tubes were vortexed and then centrifuged at 6000 rpm for 10 minutes. The supernatants were decanted and discarded. De-ionized water was then added to each tube and then vortexed again. The slurries in each tube were combined to a single tube and the contents centrifuged as before. The supernatant was decanted and the solids were washed with de-ionized water one more time. After decanting the final wash supernatant a precipitate of silver saccharinate solids remained. To the solids was added 2 ml of ethyl cellulose solution (Dow Chemical Company, Midland, Mich., Ethocel Standard 10 Premium grade, 3% w/v) and then the total content was vortexed to obtain uniform viscous opaque white slurry.

Preparation of Silver Saccharinate Layer on a Liner: the Viscous Slurry was Coated on a silicone based release liner piece with paper backing (40# bleached sulfate paper, 2.5 mil thick from LLT Bar Code & Label, Stow, Ohio) approx 4″ wide and 6″ long with the help of a Meyer rod #10. After the first coat of slurry dried, a second coat was applied to increase the coat weight. A total of 4 coats were applied and the liner was then left to air dry while being protected from light.

Adhesive preparation and coating: Into a dram vial of 20 ml capacity, was transferred ˜2 g of adhesive fluid (D380-2819, Intellicoat Technologies, UK). To dilute the adhesive, ethyl acetate (2 ml) was added. The vial contents were mixed to homogeneity and then the adhesive composition was coated onto the dried layer of silver saccharinate layer using a draw down application method (Meyer rod #10). The adhesive coating was allowed to dry to decrease the solvent content and increase the tack of the material. To accelerate drying the adhesive layer was heated briefly.

At this stage, another release liner compatible with the adhesive layer may be applied to the adhesive layer and the silver laminate construct may be stored for later use.

Preparation of Foam Made Antimicrobial with Silver Laminate Construct: a Small piece (˜1″×2″) of the laminate construct (paper liner+silver saccharinate layer+adhesive layer+liner) was cut to size and the release liner in contact with the adhesive was removed. The adhesive layer was put in contact with EVA foam of the same size (Type #2 EVA, Rubberlite Industries, Huntington, W. Va.). This formed an antimicrobial foam with silver laminate construct with a release liner still covering the silver saccharinate layer. To ensure no air entrapment, a glass test tube was rolled over the construct to act as a nip-roller. The release liner covering the AgSacc layer was peeled off without difficulty to expose the silver saccharinate layer. The layer did not exhibit any adhesive property indicating that adhesive did not leak through the slurry coating.

Antimicrobial test: In a zone of inhibition assay, the silver saccharinate foams showed a zone of inhibition 2 mm wide surrounding a disk of 8 mm dia against MRSA. Untreated foam disk showed no zone. The foam construct made with silver film laminate was found to be antimicrobial.

### Example 2

### Determination of Silver Content of the Liner with Silver Saccharinate Layer

Several small pieces of liner coated with a silver antimicrobial layer (without the adhesive) were cut from the large piece made in Example 1. Using several uncoated liner pieces of different sizes, a correlation between the weight of the liner and its area was established as follows. Weight of liner=0.0073×(Area)+0.0024

From the size of the silver coated pieces, their liner weights were estimated with the help the above correlation. The individual pieces were stripped of their silver and their silver content was calculated in ppm from FAAS results and their silver loading was estimated at ˜143±12.1 μg/cm².

The uniformity of the silver saccharinate coating was established by preparing three different coated samples and analyzing them for silver by FAAS. The results showed silver loading values of 157, 161 and 152 μg/cm² respectively. The values are quite close indicating that the coating process is consistent.

### Example 3

### Long Term Antimicrobial Efficacy of Foam Construct with Silver Laminate Construct

An EVA foam was made antimicrobial using a laminate construct made according to Example 1 was tested for long term antimicrobial efficacy in serial transfer zone of inhibition assays. Briefly, the sample from a 24 h ZOI assay was transferred to a second petri-dish coated with a fresh lawn of bacteria and incubated at 37° C. for 24 h as before. The serial transfer step was continued until clear zones were no longer seen. By this method a duration of at least 10 days was observed before the testing was terminated. In this example, the antimicrobial activity was observed for at least 10 days for foam with silver loading for ˜190 μg/cm².

### Example 4

### Effect of ETO Sterilization on Antimicrobial Foam with Silver Laminate Construct

A foam made antimicrobial with silver laminate construct, of ˜1″×2″ size was prepared as described in Example 1. The sample with silver saccharinate layer exposed was packaged in ETO permeable pouch and sent for ETO sterilization at a local facility. There was no color change after ETO sterilization.

### Example 5

### Preparation of Foam with Silver Laminate Construct—Method 2

As in Example 1, a silver saccharinate slurry was prepared by mixing 15 ml each of sodium saccharinate (0.125M) and silver nitrate (0.1M). The tube with slurry was vortexed a few minutes and then centrifuged. The supernatant was decanted and the solids rinsed three times with ethanol with centrifuging step in between. After the final ethanol rinse, (amount of residual ethanol ˜2.5 g/g of dry solids), 2 g ethyl cellulose solution in ethanol (12% w/v) was added to the wet solids and the slurry re-vortexed.

A piece of silicone release paper liner was coated with the silver saccharinate slurry and dried in ambient air for ˜1 h. A piece of liner coated with the dried silver saccharinate layer was uniformly pressed against the exposed adhesive side of a double sided tape (˜1″ width, 3M 9415 from Fralock Industries, Canoga Park, Calif.). At this point, the silver laminate construct can be stored until ready for further use. In the present example, the release liner of the double sided tape was removed and 2nd adhesive layer exposed. The exposed layer was pressed against one surface of EVA foam sheet (˜ 3/16″ thick closed cell type) to bind the silver laminate construct to the foam to form an antimicrobial foam. For antimicrobial testing, the silicone release liner covering the silver saccharinate coating was removed.

In antimicrobial testing (ZOI assay), the antimicrobial foam was found to be effective against MRSA with clear zone width ˜4 mm surrounding the sample.

### Example 6

### Antimicrobial Foam Made with Silver Saccharinate Impregnated Fabric

Silver saccharinate solids were made in a manner similar to that described in Example 5. Instead of adding ethyl cellulose solution, ethanol (10 ml) was added to the slurry. The diluted slurry was transferred to a 6″ dia petri-dish. A 3″×3″ piece of polyethylene woven fabric was soaked in the slurry for 30 seconds and blotted to remove excess liquid and dried for 30 minutes at 55° C. in an oven. A piece of the silver saccharinate impregnated fabric (˜1″×2″) piece was cut and pressed against exposed adhesive layer on one side of the double side adhesive tape. Then the adhesive side of the tape was exposed and pressed against a piece of EVA foam of same size. Due to silver saccharinate particles binding tightly to the fabric, there was no rub off of the silver salt.

In antimicrobial tests, the antimicrobial foam with woven silver saccharinate impregnated fabric was found to be antimicrobial against MRSA with inhibition zone width of ˜6 mm. Sterilization by ETO did not affect the color of the silver impregnated fabric which remained opaque white.

### Example 7

### Preparation of Silver Laminate Construct and Application to Foam

Silver saccharinate slurry with the following composition was prepared in a manner described in Example 1.

| Silver Saccharinate | 12.3% w/w |
|---|---|
| Ethyl cellulose | 8.7% |
| Ethanol | 79.0% |

The slurry was coated on an acrylic liner without siliconization, to form the antimicrobial layer and the adhesive (same as in example 1 but without dilution) was coated on a silicone liner using draw down technique to form the adhesive layer. After both coatings were dried to form layers, the exposed surface of the antimicrobial layer was contacted with the exposed surface of the adhesive layer, so that the liners were on the outside of the laminate. To a piece of EVA foam, the silver laminate construct was applied with the adhesive layer contacting the foam. The release liners worked as intended, meaning the release liner on the adhesive layer released correctly when bonding to the foam and the release liner covering the silver saccharinate layer peeled off smoothly. The silver saccharinate layer was non-tacky to feel.

### Example 8

### Preparation of Silver Laminate Construct and Applying it to Foam

Silver saccharinate comprising composition was modified for this example. The modified composition was as follows.

| Silver Saccharinate | 12.5% w/w |
|---|---|
| Ethyl cellulose | 7.0% |
| Ethanol | 80.5% |

To 100 g of silver saccharinate composition above, 2.5 g of adhesive (Durotak 387-2051 from National Starch) was directly added and blended in. The modified silver containing composition with small amount of adhesive was coated on acrylic type non-silicone liner and dried to form an antimicrobial layer. On a silicone type liner, a coating of adhesive was applied by draw down method and dried to form an adhesive layer. The exposed surface of the antimicrobial layer was contacted with the exposed surface of the adhesive layer, so that the liners were on the outside of the laminate. The antimicrobial foam was made from EVA foam as described in Example 7. The release liners worked as intended. The silver saccharinate coating side was barely tacky to feel.

### Example 9

In a modification of the silver laminate construct of example 8, an adhesive layer was directly applied over the silver saccharinate layer and then a silicone liner was applied. An antimicrobial foam was made by attaching the silver laminate construct to a foam. Once again the silver saccharinate layer was barely tacky to feel.

### Example 10

### ETO and Light Resistance of Silver Laminate Construct of Example 8

A ˜2″×1″ antimicrobial foam was made according to Example 8 was sterilized by ETO at a local facility. There was no color change after ETO sterilization.

Another piece of antimicrobial foam was exposed continuously to light from a 60 W incandescent lamp at a distance of ˜1.5° for 1 week. Barely discernable color change to yellow was observed. But the color change was uniform over the entire surface and aesthetically acceptable.

### Example 11

### Silver-CHG Laminate Construct

To 10 g of AgSacc slurry similar to that in example 8 in a dram vial was added 0.25 g of adhesive (Durotak 387-2051, National Starch) followed by 0.05 ml of 20% solution of Chlorohexidine gluconate (CHG) (Spectrum Chemicals Corp. Gardena, Calif.). The contents were mixed well on a vortex mixer. Using Meyer rod #10 the slurry was applied on a piece of paper-based silicone release liner (3″×1″) and was allowed to dry to form the antimicrobial layer. On top of the antimicrobial layer was applied a coating of adhesive (Intellicoat Technologies D380-2091) and solvent was allowed to escape to form an adhesive layer. An EVA foam piece was pressed on the adhesive layer. The liner was removed to expose the antimicrobial layer. In ZOI assay against MRSA, the antimicrobial foam was found to antimicrobial and showed slightly larger clear zone than the construct of Example 8 indicating a synergy between silver and CHG.

### Example 12

### Direct Coating of Silver Saccharinate on EVA Foam

In a 50 ml polypropylene conical bottom tube, aqueous sodium saccharinate solution (20 ml, 0.125M) was pipetted followed by the addition of aqueous silver nitrate solution (20 ml, 0.1M) under vortexing to yield a milky white suspension of silver saccharinate. The suspension was centrifuged three times. After each centrifuge, the supernatant was decanted and deionized water (10 ml) was added to the solids and vortexed. After the third centrifugation and decanting, the wet solids were composed of water and silver saccharinate in a weight ratio of ˜2:1. A small amount of deionized water (2 ml) was added to the wet solids to yield a milky white paste.

With the help of a transfer pipette, the paste was spread on one edge (short dimension) of a 1″×4″ size piece of EVA foam (Type #2 EVA, Rubberlite Industries, Huntington, W. Va.). Using a #10 Meyer rod, the paste was spread on the foam to form a thin wet film of the paste material. The foam was air dried for 5 minutes and then transferred to an oven set at 55° C. and dried for an additional 75 minutes. Under microscopic examination, the silver saccharinate solids were uniformly coated on the foam. However, in handling the sample, some rub off of the silver saccharinate was observed.

### Test for Antimicrobial Activity

The silver saccharinate coated foam was tested for anti-microbial property by standard zone of inhibition (ZOI) assay. Briefly, a disk (˜1 cm dia) was cut from the foam piece and placed on freshly laid lawn of an overnight fresh culture of Staphylococcus aureus on an Meuller Hinton Agar (MHA) plate. Untreated foam disk and a hydrated disk of SilvaSorb sheet was used as negative and positive controls, respectively. The MHA plate was incubated at 37° C. overnight and clear zones surrounding each sample disk was measured and recorded.

To estimate the sustained release character of the coated foam, the same samples were subjected to a serial transfer test. The disks from ZOI assay after day 1 were transferred to another MHA plate covered with fresh lawn of bacteria. The plate was incubated as before and the procedure repeated each day until clear zone surrounding the silver bearing sample was no longer observed. The number of days of serial transfer of sample disk was recorded as the duration over which the foam was efficacious.

The silver foam sample showed clear zone against Staphylococcus aureus in ZOI assay clearly indicating anti-microbial activity. In the serial transfer test, the anti-microbial efficacy was observed for 3 days.

### Test for Resistance to Discoloration

A 1″×1″ foam piece of silver saccharinate coated foam prepared above was placed on lab bench and exposed to ambient lab light for 24 h and examined for discoloration. The foam piece showed little discoloration with only traces of faint grey color.

### Test for Effect of ETO Sterilization

Another piece of foam (1″×1″) was sealed in a moisture permeable paper pouch and sent out for ETO sterilization at a local facility in Portland, Oreg. area. The sample was examined and compared with an untreated foam piece. After ETO treatment, there was practically no difference in color of the silver treated and untreated foam. See FIG. 5. This is quite remarkable considering most silver containing devices will discolor rapidly during ETO sterilization due to the reduction of silver salts to elemental silver.

### Example 13

### Direct Coating of Silver Saccharinate on Eva Foam

In a 50 ml polypropylene conical bottom tube, aqueous Tween 20 solution (15 ml, 16.7 gm/l) and aqueous sodium saccharinate solution (15 ml, 0.125M) were pipetted successively followed by the addition of aqueous silver nitrate solution (15 ml, 0.1M) under vortexing to yield a milky white suspension of silver saccharinate. The suspension was centrifuged three times. After each centrifuge, the supernatant was decanted and deionized water (10 ml) was added to the solids and vortexed. After the third centrifugation, the wet solids were water and silver saccharinate in a weight ratio of ˜2:1.

A small amount of deionized water (2 ml) was added to the wet solids to yield a milky white paste.

The milky paste was coated on the foam piece (2″×8″) as described in Example 12. Several foam pieces of 1″×1″ size were cut from the foam, packaged and sterilized by ETO. No discoloration was observed in the sterilized samples. As in Example 12, some rub off of the active silver compound was observed.

### Test for Skin Staining

Sterilized foam pieces from Example 13 were used in the test. The silver saccharinate coated foam pieces were tested on human skin. Four human subjects were used, one for each day, length of exposure, tested, and two locations on each subject were tested. On two places, either each forearm or on the back were applied 0.5″×0.5″ square pieces of silver coated foam, with the silver coating contacting the skin, and untreated foam (control). Each test sample on skin was affixed in place with the help of Opsite® Flexigrid® thin film dressing from Smith & Nephew Company. In a pre-determined manner, the samples were removed from the subject's skin and the area under the sample was examined for staining by silver. No staining due to silver saccharinate coated foam was seen on any subject after day 1, day 2, day 3 and day 7 was observed.

### Test for Antimicrobial Activity

Foam samples were prepared according to the method described in Example 12 except they were not sterilized. This test for antimicrobial activity was essentially a serial transfer ZOI assay but with modification. The samples for this assay were in the form of 1″×1″ squares with an 8 mm hole in the center. The rationale for preparing the sample in manner was to mimic the foam element present in the Huber needle device. The samples were laid on MHA plate streaked with two lines of Methicillin resistant Staphylococcus aureus (ATCC 33591) at right angle to each other and intersecting in the center of the MHA plate. The samples with coating side contacting agar surface were laid such that the hole center and the point of intersection of streaks were coincident. Coated samples were used in triplicate and one untreated foam sample served as negative control. SilvaSorb hemispherical disk was laid over one streak of bacteria as positive control. The plates were incubated at 37° C. for 24 h. Due to the presence of silver on the treated sample, no bacterial growth was seen inside of the hole. But, the hole inside of the untreated control showed growth. Next day, the samples were transferred to a second MHA plate made identically as before and incubated as before. The procedure was repeated each day until the treated sample started to show bacterial growth inside the hole. The final results showed the antimicrobial activity due to the treated sample lasted 10 days.

### Example 14

Silver saccharinate slurry was prepared by the procedure described in Example 1 except, the total amount of silver nitrate solution (0.1M) was 80 ml and after the final aqueous rinse, an ethanol rinse was attempted. The supernatant ethanol was decanted and the wet cake of silver salt (solids content ˜50% w/w) was set aside.

In a dram vial (˜22 ml capacity), 1.13 g ethyl cellulose (Ethocel Std 100, Dow Chemical) was dissolved in ethyl acetate to yield 13.2 g of clear viscous solution. To this solution, 1.8 g of silver saccharinate wet cake was added and vortexed to obtain a uniform white viscous slurry. To the slurry, 2.14 g adhesive solution (Aeroset 1920-Z52, Ashland Chemical Company) was added to obtain silver antimicrobial composition with slight tack.

In a similar dram vial, adhesive composition was prepared by mixing 10 g of 20% w/w acrylic polymer (Avalure AC315, Lubrizol Corp) solution in ethyl acetate and 5 g adhesive solution (Aeroset 1920-Z52, Ashland Chemical Company). On several strips (1″×4″) of silicone release paper liner (#40 bleached sulfate paper, 2.5 mil thick from LLT Bar Code & Label, Stow, Ohio) first adhesive composition coat was applied using Meyer rod #20 and dried for 30 seconds with a household hair dryer on high setting. A second adhesive composition coat was similarly applied over the adhesive layer and dried in an oven at 85 C for 3 minutes. On several strips of identical size liner made of a poly coated brown color paper liner (#72 RF-7000-33, Rayven Inc, St. Paul, Minn.), the silver saccharinate antimicrobial composition made above was coated using Meyer rod #20 and the antimicrobial composition dried at 85 C for one minute in the oven to form an antimicrobial layer.

The two liner strips with an adhesive layer and silver saccharinate antimicrobial layer respectively were aligned and then pressed together (with the liners on the outside) with the help of a rolling pin by rolling it several times. The laminate construct was complete. To demonstrate differential release, the liner on the adhesive side was peeled off easily by grabbing at a corner of the strip (Note for differential release to be correct, only the liner should come off without any portion of adhesive layer or the underlying silver coating de-laminating). The exposed adhesive side was pressed against a similarly sized EVA foam strip and pressure was exerted by moving a rolling pin over it several times. The brown paper liner was grabbed at a corner and peeled readily off leaving behind an intact silver saccharinate film bonded to the EVA foam (Note the release is deemed successful only if no portion of the silver film bonded to the foam comes unglued). Because both liners released cleanly and correctly without compromising the silver film, the differential release feature of the laminate construct was demonstrated successfully.

### Example 15

### Aging Effect on Laminate Construct

Silver saccharinate antimicrobial compositions and adhesive compositions were made as in Example 14 and applied on the same liners except the dry times for 2^{nd} adhesive layer and silver layer were 6 minutes and 3 minutes respectively with the oven temperature remaining same. Three laminate strips were made, applied on EVA foam and each time showed consistent differential release of liners. A 4^{th} laminate strip was aged in an oven at 40 C for 8 days and tested for differential release. Just like a freshly made laminate the aged strip performed as expected with respect to differential release to obtain EVA foam strip with silver film bonded on one side. On an average all silver films on foam strips made in this example exhibited slight tack at the surface.

### Example 16

### Varying the Amount of Adhesive in Silver Antimicrobial Layer and in the Adhesive Layer to Change the Levels of Tack

The following solutions were prepared:

Solution A: 1.13 g ethyl cellulose (Ethocel Std 100, Dow Chemical) was dissolved in ethyl acetate to yield 13.2 g of clear viscous solution. To this solution, 1.8 g of silver saccharinate wet cake was added and vortexed to obtain a uniform white viscous slurry.
Solution B: 15 g of 20% w/w acrylic polymer (Avalure AC315, Lubrizol Corp) solution in ethyl acetate was prepared by dissolving appropriate amount of the polymer in the solvent.
Solution C: Adhesive solution (Aeroset 1920-Z52)

The following antimicrobial compositions and adhesive composition were prepared (in parts by weight proportion):

| Solution type | Solution A | Solution B | Solution C |
|---|---|---|---|
| Silver coating Ag-1 | 7 | | 1 |
| Silver Coating Ag-2 | 11 | | 1 |
| Silver Coating Ag-3 | 9 | | 1 |
| Adhesive Coating Ad-1 | | 2 | 1 |
| Adhesive Coating Ad-2 | | 3 | 1 |

Same liners as in the Example 14 were used in this example. The application, the drying conditions and the construction of EVA foam strips were similar to Example 15. The observations of differential release and the feel of the silver film on the foam were recorded and are listed in the table below.

| Test | Silver | Adhesive | Observation of differential release |
|---|---|---|---|
| No. | coat | coat | and EVA foam with silver film |
| 1 | Ag-1 | Ad-1 | Good differential release, the silver film |
| | | | bonded nicely to the foam, slight tack |
| 2 | Ag-2 | Ad-2 | Poor differential release; Foam strip could not |
| | | | be constructed |
| 3 | Ag-3 | Ad-2 | Good differential release; Nice foam strip with |
| | | | less tack than Test no. 1 |

### Example 17

Silver antimicrobial composition Ag-3 (see Example 16) was prepared and two adhesive compositions (Ad-4 & Ad-5 respectively) with Solutions B and C (see Example 16) in ratios of 3.5/1 and 4.0/1 were prepared. The adhesive composition was coated with the help of Meyer rod #20 on #40CK liner (TaylorMade Labels Inc) and silver antimicrobial composition using Meyer rod #40 on the brown polycoated #72 release liner. Drying was carried out at 85° C. to form layers. The laminate constructs were examined for differential release by constructing EVA foam strip with silver film. Note laminate bonding to EVA foam was under pressure exerted by rolling a test tube under hand pressure over the laminate. The results are summarized below.

| | | Drying Duration | | |
|---|---|---|---|---|
| Silver | Adhesive | (min) | Results | |
| comp | comp | Ad:Ag | Diff. Rel. | Observations |
| Ag-3 | Ad-4 | 3:2 | Pass | Laminate construct showed good |
| | | | | differential release, less tacky feel to |
| | | | | silver film |
| Ag-3 | Ad-4 | 3:2 | Pass | Good release, less tacky feel to silver |
| | | | | film on foam |
| Ag-3 | Ad-5 | 3:2 | Fail | Inconsistent differential release |
| Ag-3 | Ad-5 | 6:3 | Fail | Increasing drying did not help. Poor |
| | | | | differential release. Failed |
| | | | | experiment |
| Ag-3 | Ad-5 | 6:3 | Fail | Imperfect release even after testing |
| | | | | another liner (GMC) for adhesive |
| Ag-3 | Ag-5 | 6:3 | Fail | A wringer was used to press the liner |
| | | | | in this experiment. The adhesive was |
| | | | | smoothed very well, but the two |
| | | | | liners did not attach. |
| Ag-3 | Ad-4 | 10:2 | Fail | Extended drying for adhesive did not |
| | | | | improve differential release. Failed |
| | | | | experiment |

### Example 18

### Laminate Constructs Made with Higher Drying Temperature and in Wide Format

Laminates were constructed by applying silver antimicrobial composition Ag-3 (see Example 16) using Meyer rod #40 and adhesive composition Ad-4 (see Example 17) using Meyer rod #20. Same liner as in Example 17 was used for silver coat but the liner for adhesive in this example was #42 CK release liner (identical to #40 CK except it is slightly heavy and sourced from the same vendor). The drying temperature was increased to 100-105° C. to ensure all toluene was removed from the layers. The width of the layers was increased (Three samples each at 1″, 2″ and one at 3″ width, the length was ˜4″) to observe if differential release was still taking place correctly despite the increased overall area On production scale, the width may be greater. In addition, pressure was exerted on the laminate-foam strip construct by passing it through a wringer (used in wringing shop rags) after initial test tube roll press. The results are tabulated below.

| | | | Drying | | |
|---|---|---|---|---|---|
| | | | duration | | |
| Width | Silver | Adhesive | (min) | Diff | |
| (inch) | comp | comp | Ad:Ag | Rel. | Observations |
| 1″ | Ag-3 | Ad-4 | 3:2 | All | Sample 1 good release |
| | | | | Pass | Sample 2 showed good release but, some |
| | | | | | bubbling on adhesive side due to slight |
| | | | | | overheating during drying. Sample 3 good |
| | | | | | release but required additional wait period |
| | | | | | before release occurred |
| 2″ | Ag-3 | Ad-4 | 3:2 | Mixed | ⅔ samples showed good release. Sample |
| | | | | | that failed showed uneven adhesive |
| | | | | | bonding (due to improper pressure |
| | | | | | application) |
| 3″ | Ag-3 | Ad-4 | 3:2 | Pass | Very good result, because there are no |
| | | | | | complication when the CK liner was |
| | | | | | peeled off. |

Note a few addition laminate constructs were made as above except the adhesive drying was attempted at 132° C. As a result of high temperature exposure, the adhesive layer seemed to lose its tack and did not bond well with silver coating and therefore the laminate construct did not form well.

### Example 19

### Test Silver Coating for Blocking Resistance

In large scale production of the silver laminate construct, the silver antimicrobial composition will be applied on one side of the liner material that will be rolled up. It is important that in the roll form the silver antimicrobial layer does not inadvertently stick to the backside of the liner.

Silver antimicrobial composition (Ag-3, see Example 16) was applied on the brown paper liner (#72 Polycoated RF-7000-33, Rayven Inc) squares 3″×3″ size (No. of samples: 6) with the help of Meyer rod #40. Each paper liner piece was dried at 100-105° C. and cooled to room temperature. The sample pieces were stacked one on top of each other and the stack placed on a flat surface e.g. petri-dish or glass plate and kept in an oven at 32-38° C. under about 1 kg weight for 10 minutes. The stack was removed and examined to see if each piece remained separate from the others i.e. not stick under weight. Each piece readily came apart from the stack. Therefore, the silver coating exhibited decent block resistance.

### Example 20

The laminate construct samples in this example were made by using another adhesive (Aroset S390, Ashland Chemical Company) in adhesive composition Ad-1 and Ad-2 (see Example 16). The silver antimicrobial composition Ag-3 (see Example 16) was still made using the old adhesive (Aroset 1920-Z52). The respective liners, the drying conditions for the coatings and the lamination to EVA foam conditions were same as those used in Example 18. The results of differential release testing are tabulated below.

| | | | Drying | | |
|---|---|---|---|---|---|
| length | Silver | | Duration | | |
| (inch) | comp | Adhesive | (min) Ad:Ag | Diff. Rel. | Observations |
| 1″ | Ag-3 | Ad-1* | 3:2 | Pass | Sample 1 - Still a bit tacky, this |
| | | | | | may be because the 1920-Z52 |
| | | | | | adhesive in silver coat; Sample 2 - |
| | | | | | Freshly made sample easy to |
| | | | | | peel but still tacky; Sample 3 - 8 |
| | | | | | days aged (40° C.) peeled off |
| | | | | | easy; Sample 4 -14 days aged |
| | | | | | peeled off easy. All samples |
| | | | | | showed good diff. release |
| 1″ | Ag-3 | Ad-2* | 3:2 | Pass | Not enough adhesive on the CK |
| | | | | | liner side, because the new |
| | | | | | adhesive is less tacky. Did not |
| | | | | | bond well to the foam strip |
| 3″ | Ag-3 | Ad-1 | 3:2 | Pass | Samples 1 & 2 - Fresh made |
| | | | | | samples showed good diff. |
| | | | | | release. Sample 3 - aged for 14 |
| | | | | | days also peeled off easy. Good |
| | | | | | release though a bit tacky. |

*Made with Aroset S390 adhesive; Meyer rod #40 for silver & # 20 for adhesive

### Example 21

### Laminate Constructs

Stock solutions of ethyl cellulose (Ethocel Std 100, Dow Chemical Co.) in ethyl acetate (˜8.56% w/w) and of Avalure AC315 in ethyl acetate (20% w/w) were prepared by dissolving the respective solids in the solvent under slight warming. Wet cake of silver saccharinate was prepared by precipitating the silver salt by mixing silver nitrate solution (46 ml, 0.15M) into sodium saccharinate solution (70 ml, 0.125M), rinsing the precipitate first with deionized water and then ethanol. After discarding the ethanol, wet cake (approximately 2 gm) was obtained (˜50% w/w solids).

To the wet cake, 14.1 gm ethyl cellulose solution was added, vortexed to homogeneity to yield silver saccharinate slurry. The silver coating solution was made by mixing the silver slurry and Aroset S390 adhesive in 4/1 ratio. Additional silver coating solutions were made by employing 6/1, 7.5/1 w/w ratio. In a separate dram vial, Avalure AC315 solution and Aroset S390 adhesive were mixed in 2/1 ratio.

Using Meyer rods #40 and #20, silver antimicrobial layer and adhesive layer were formed on the same liner pair as in Example 18 to prepare several 1″ wide, 4″ long laminate constructs. Pressure application method employed to laminate coatings was similar to Example 18. The differential release was qualitatively examined and silver film quality on EVA foam strips was evaluated. The results are tabulated below.

| Silver | Avalure | | | | |
|---|---|---|---|---|---|
| slurry | AC315 | Pressure | Drying | | |
| to | soln to | Application, | duration | | |
| S390 | S390 | Liner | (min) | | |
| ratio | ratio | type | Ad:Ag | Result | Observations |
| 4:1 | 2:1 | Wringer, | 3:2 | Pass | The brown liner peeled off |
| | | Tube, CK | | | easily, but the silver film was too |
| | | liner #42 | | | tacky. |
| 6:1 | 2:1 | Wringer, | 3:2 | Pass | Both liners peeled off easily, but |
| | | Tube, CK | | | silver film was slightly tacky |
| | | liner #42 | | | |
| 7.5:1 | 2:1 | Wringer, | 3:2 | Pass | Sample 1 - Easy peeling on both |
| | | Tube, CK | | | sides, and very little tackiness. |
| | | liner #42 | | | Sample 2 - 7 days aging, easy |
| | | | | | peel off of both liners. Sample 3 - |
| | | | | | 14 days aging, easy peel off of |
| | | | | | both liners |

Note
additional EVA foam strips 3″ wide were made using silver coating soln (7.5/1) and adhesive soln (2/1) above. The increased width did not affect differential release with both liners peeling off readily.

### Example 22

### Laminate Constructs with Different Liners on Adhesive Side

Silver coating solution (7.5/1) and adhesive coating solution (2/1) from Example 21 was re-used in this example. The same respective Meyer rods as in Example 21 were used to form coatings and same drying conditions and pressure exertion conditions to form laminate constructs were employed. However, the adhesive coating was formed on two film liners—#38 silicone liner and Loparex film liner each with silicone release layer. The laminates made were applied to EVA foam strips (˜1″×4″) and tested for differential release. The results obtained are tabulated below.

| | | | Drying | | |
|---|---|---|---|---|---|
| | | Pressure | Duration | | |
| Silver | Adhesive | Application, | (min) | | |
| comp | comp | Liner type | Ad:Ag | Diff. Rel. | Observations |
| 7.5:1 | 2:1 | Wringer, | 3:2 | Pass | Fair differential release, but |
| | | tube. #38 | | | not as well as CK #42 paper |
| | | silicone liner | | | liner. |
| 7.5:1 | 2:1 | Wringer, | 3:2 | Fail | Accidental de-lamination of |
| | | tube, | | | silver coating during the |
| | | Loparex | | | construct passage through the |
| | | Film Liner | | | wringer. Undesirable diff. |
| | | | | | release. Too much pressure |
| | | | | | from wringer may cause the |
| | | | | | Loparex film liner not to |
| | | | | | release from adhesive layer. |
| 7.5:1 | 2:1 | No wringer, | 3:2 | Pass | Without the wringer, the |
| | | tube, | | | Loparex flm detached from |
| | | Loparex | | | the adhesive layer due less |
| | | Film Liner | | | pressure application. Proper |
| | | | | | diff. release. |
| 7.5:1 | 2:1 | Wringer, | 3:2 | Pass | Diff. release of #38 liner but |
| | | tube. #38 | | | adhesive side was not tacky. |
| | | silicone liner | | | |
| 7.5:1 | 2:1 | Wringer, | 3:2 | Fail | 24 h at 40° C. aging affected |
| | | tube. #38 | | | diff, release adversely. But |
| | | silicone liner | | | fresh sample okay. |
| 7.5:1 | 2:1 | Wringer, | 3:2 | Fail | 24 h at 40° C. affected the |
| | | tube. #38 | | | bonding to foam. Adhesive |
| | | silicone liner | | | side not as tacky. |

### Example 23

### Laminate Constructs with Avalure AC315 Replacing Ethocel in Silver Coating

Though both Ethocel and Avalure AC315 have good film forming property, the films of Ethocel are somewhat fragile. This example shows the results of laminate constructs made by replacing Ethocel with Avalure AC315 in the silver coating.

Silver antimicrobial composition: Silver nitrate solution (23 ml, 0.15M) and sodium saccharinate solution (35 ml, 0.125M) were mixed to precipitate silver saccharinate. The precipitate was rinsed with deionized water and ethanol respectively. The resulting wet cake was mixed in Avalure AC315 solution in ethyl acetate (20% w/w, 14 g) and vortexed to homogeneity. The silver slurry above and Aroset S390 adhesive solution were mixed in 9.5/0.5 ratio to obtain the silver coating solution. Meyer rod #40 was used to coat the antimicrobial composition on the same polycoated (#72) brown paper liner.
adhesive composition: The Avalure AC315 solution in ethyl acetate (20% w/w) and Aroset S390 adhesive were mixed in 2/1 ratio to homogeneity. Meyer rod #20 was used to apply coating on #38 silicone release. The laminates were 1″×4″ in size and applied on EVA foam under same pressure conditions as in Example 22. The drying temperature was 105-110° C.

| | Drying | | |
|---|---|---|---|
| Pressure | time | Diff. | |
| application | (min) Ad:Ag | Rel | Observations |
| Tube, | 3:3 | Pass | Lack of tack on silver coating. Only partial attachment to |
| Wringer | | | the foam. Need to improve |
| Tube, | 3:3 | Pass | Good diff. release. Good bonding to the foam. Avalure in |
| Wringer | | | silver film impart greater flexibility and stretchability to |
| | | | the film than Ethocel |
| Tube, | 3:2 | Pass | Small amount of adhesive in silver coating solution |
| Wringer | | | improves spreadability of the wet coating on the liner. |
| | | | Good diff. release and bonding to the foam. |
| Tube, | 3:2 | Pass | Good diff. release and ready bonding to the foam. Silver |
| Wringer | | | film could be bonded to other curved surfaces without |
| | | | film breaking. |
| Tube, | 3:2 | Pass | Good diff. release. Applied to Silver PU foam prototype |
| Wringer | | | instead of EVA foam. |
| Tube, | 3:2 | Pass | Good diff. release upon application to masking tape. The |
| Wringer | | | masking tape was wrapped around a test tube without |
| | | | breaking the silver film. |

### Example 24

### Effect of Aging on Laminate Constructs

Several silver laminate constructs (1″ wide and 4″ long) made in Example 23 (drying times of adhesive and silver layers 3 min and 2 min respectively) were placed in oven at 40° C. for up to 14 days to simulate ageing. The aim of the test was to see if differential release was maintained in the laminate even after accelerated ageing.

At the end of aging duration (7 or 14 days), the liner on adhesive side was peeled off and the laminate strip bonded to EVA foam. After applying pressure under conditions similar to Example 23, the brown paper liner was peeled off to expose silver film. Regardless of the aging duration, the laminate construct performed as intended i.e. exhibited correct and smooth differential release; bonded readily and uniformly to the foam. Therefore, laminate constructs based on Avalure AC315 in both silver and adhesive coats exhibited reasonable shelf life, a requirement for device production on large scale.

### Example 25

### Resistance to Blocking of Silver Antimicrobial Layer

600 g of silver coating slurry consisting of 12% w/w silver saccharinate, 20% w/w Avalure AC315 in ethyl acetate was prepared by following the procedure described in Example 23 with proportionate increase in the ingredients used. It was mixed in with S390 adhesive in 9.5/0.5 ratio to obtain silver coating solution.

Six strips (3″×4.5″) of brown paper liner (#72, RF-7000-33, Rayven Inc. St. Paul, Minn.) were coated with silver coating solution using Meyer rod #40. After drying them at 105-110° C., they were cooled to room temperature, stacked in a pile and placed on a flat surface (petri-dish) under 1 kg weight (Nalgene bottle filled with 1 liter water) in an oven at 40° C. overnight.

Thereafter, the stack was removed, cooled to temperature and examined to see if individual strip could be removed cleanly. We observed each strip came off from the stack with no sign of adhesion between the samples. Clearly, this showed the silver coating possessed excellent resistance to blocking, needed for large scale production of the laminate construct.

### Example 26

### Durability of Silver Antimicrobial Layer Under Wet Wipe Conditions

As described in the specification, the silver film laminate can be used to render a variety of surfaces antimicrobial. It is also important, however, to have the antimicrobial effect to be durable. This example describes a wet wipe test conducted on a glass slide coated with silver antimicrobial layer and the strong antimicrobial efficacy demonstrated after a large number of wet wipes.

One of the silver antimicrobial layer brown liner sample from Example 25 (after test completion) was laminated with adhesive (the adhesive formula, coating, drying and lamination conditions same as in Example 24). The laminate thus obtained was bonded to a clean glass slide (1″ wide 3″ long, Fisher Scientific) instead of EVA foam. The brown liner was removed to expose silver antimicrobial layer.

The wet wipe test of the silver antimicrobial layer bonded to the glass was carried out as follows: A soap solution was prepared by dissolving a drop of dish soap (Dove® dishwashing soap) in 25 ml deionized water. A paper sheet (Kim-wipe brand, Kimberly-Clark) wetted with dish water was used to wipe over the silver film followed by wiping with paper sheet wetted by water. The silver antimicrobial layer was dried with air. These steps completed one wipe cycle. Each day 10 wipe cycles were performed. Each day after the wipe test, the silver antimicrobial layer was examined for signs of de-bonding and for any color change. Over the duration of the test after the wipe portion, the glass slide was left on the bench under routine lab light exposure. In all, 200 wipe cycles over 20 days were done. At the end of 20 days, antimicrobial efficacy of the silver film was tested by zone of inhibition (ZOI) assay against a slate of common microorganisms. The results indicated potent activity from the silver antimicrobial layer implying that ionic silver was still releasing from the antimicrobial layer surface.

### Example 27

### Construction of Device Prototypes with Antimicrobial Laminate Construct

Several silver antimicrobial layer brown paper liner strips (3″×4.5″) from Example 25 were used in this example. As needed the liner strips were cut into thin strips or as circles for constructing device prototypes as described below.

### Device 1: Antimicrobial Handle Bar

The idea was to have an antimicrobial barrier on the handle bar surface that can provide long lasting protection. In this case, a glass test tube was used to simulate a handle bar. 1″ wide strips of a preformed antimicrobial layer were cut and pressed against one exposed adhesive side of a 1″ wide double sided adhesive tape (3M Type 9415). Next, the 2^{nd} adhesive side was exposed by peeling off the liner. The exposed side of the tape was pressed against the tube surface and the tape was wound around to create helix pattern. Finally, the brown paper liner was removed to expose silver antimicrobial layer now bonded to the glass test tube surface by double sided tape.

### Device 2: Antimicrobial Handle Bar

One of the brown paper liner strips from Example 25 was laminated with adhesive layer (the adhesive composition, coating, drying and lamination conditions same as in Example 24). The laminate construct was cut into thin strips 1″ wide. The #42CK paper liner was removed to expose the adhesive side. Carefully, the adhesive side was pressed against glass test tube surface and wound around in helix pattern. The brown paper liner was removed to expose the silver film.

### Device 3: Antimicrobial Protection Device for IV Access Device

This device offers protection against infection risk associated with IV access devices, the simplest of which is IV drip. A 1″ dia circle was cut from an antimicrobial laminate construct of size 3″×4.5″. The adhesive layer was exposed and pressed on a 1″ dia premade antimicrobial silver foam (from AcryMed Inc). The brown paper liner was removed to expose the silver antimicrobial layer. A small hole was punched in the center of the round device and a slit was cut from the outer edge of the circle to the inner hole to complete device prototype construction.

A modification of the device would be to press silver coated brown liner against one adhesive side of a double sided tape, then expose the 2^{nd} adhesive side and bond it to foam (of same size and shape) that may or may not contain an antimicrobial agent.

### Example 28

### CHG Containing Laminate Construct

To 4 gm of Avalure AC315 polymer solution in ethyl acetate (20% w/w) in a dram vial, 1 gm chlorohexidine gluconate (Spectrum Chemical Co. 20% solution in ethanol) was added and vortexed to homogeneity. Using Meyer rod #40, the antimicrobial composition was coated on brown paper liner (Example 25) and dried at 105-110° C. for 3 minutes. Similarly adhesive composition (See Example 23) gave a coating upon drying for 2 minutes at 105-110 C on #42 CK paper liner with the help of Meyer rod #20. The laminate was constructed by pressing the two liners together. The adhesive layer was exposed and EVA foam piece was laminated to obtain foam with CHG bearing antimicrobial layer.

In ZOI assay, foam disks (8 mm dia) showed good antimicrobial activity against MRSA and Pseudomonas aeruginosa. However, in serial transfer assay, no antimicrobial activity was found after one day. There are methods such as encapsulation, entrapment in microspheres to extend CHG release by slowing down the diffusion of CHG from the film.

### Example 29

### Uniformity of Silver Distribution in the Antimicrobial Layer

The aim of the test was to show that the composition and the coating method (by Meyer rod) allowed us to deposit silver uniformly over 1″×4.5″ liner strip.

Two brown paper liner strips (1″×4.5″) were coated with silver antimicrobial composition using Meyer rod #40 and the antimicrobial layer was dried at 110° C. for 2 minutes. Two sets of pieces of silver antimicrobial layer liners with 1 cm×1 cm, 1 cm×2 cm, 1 cm×3 cm and 1 cm×4 cm sized pieces were cut. The liners were stripped of silver and analyzed for silver by Varian 220FS atomic absorption spectrometer. The results of silver analyses are tabulated below.

| Sample Size | Sample Set A Ag in ug/cm2 | Sample Set B Ag in ug/cm2 |
|---|---|---|
| 1 cm × 1 cm | 291.1 | 248.8 |
| 1 cm × 2 cm | 285.0 | 302.1 |
| 1 cm × 3 cm | 320.3 | 282.3 |
| 1 cm × 4 cm | 337.0 | 272.6 |

The data show a uniform distribution of silver showing the silver antimicrobial composition preparation and the method of coating for the prototypes is robust.

### Example 30

### Variation in the Amount of Silver in the Antimicrobial Composition

In this example, we varied the thickness of wet coating of silver antimicrobial composition deposited on the brown paper liner by varying Meyer rods. By selecting the rods with different numbers e.g. 10, 20, 30 etc, the wet coating thickness was adjusted.

The silver antimicrobial composition (with adhesive S390 mixed in) employed was from the same 600 g batch used in Example 25. The solution was coated on 3″×4.5″ sized brown paper liner pieces using 5 different Meyer rod types—#10, #20, #30, #40 and #50, dried in oven at 110° C. for 2 minutes to form an antimicrobial layer. Liner samples made were cut into 1″×1″ pieces and submitted for silver analysis (n=4) by FAAS. The silver analysis results are listed below.

| | Avg. Silver content |
|---|---|
| Meyer Rod # | (ug/cm2) |
| 10 | 34.47 ± 1.43 |
| 20 | 57.40 ± 2.64 |
| 30 | 86.20 ± 2.85 |
| 40 | 103.90 ± 0.26 |
| 50 | 127.50 ± 5.38 |

Averages shown with +/− one std dev.

Because the rod numbers correspond to proportionate wet (or dry) thicknesses, the rod numbers and the silver content values reflect a linear relation. Thus, the amount of silver can be varied by simply varying the wet thickness of the silver coating solution being coated.

### Example 31

### Variation in the Amount of Silver by Varying the Silver Content of the Antimicrobial Composition

By varying the amount of silver saccharinate in the silver antimicrobial composition, we prepared samples solutions with silver saccharinate contents of 1.5%, 3.0%, 4.5%, 6.0%, 7.5% and 9.0%. In all the solutions prepared the weight ratio of silver salt to Avalure AC315 was kept constant and the % of adhesive in the antimicrobial composition was held at 5% w/w.

With the help of #40 Meyer rod sample antimicrobial composition were coated on the brown paper liner (2″×4.5″); coatings dried at 110° C. for 2 minutes. Each liner sample was cut in four 1″×1″ square pieces and the pieces submitted for silver analysis. The silver content values for antimicrobial layer made from antimicrobial compositions of varying silver concentrations are presented below.

| Silver Saccharinate % | |
|---|---|
| In Silver antimicrobial | |
| composition* | Silver Content (ug/cm2) |
| 1.5 | 33.3 ± 2.00 |
| 3.0 | 66.40 ± 3.84 |
| 4.5 | 108.75 ± 2.20 |
| 6.0 | 146.10 ± 7.46 |
| 7.5 | 190.45 ± 20.46 |
| 9.0 | 252.93 ± 33.55 |

*Constant wet (or dry) thickness

### Example 32

### Construct Made Using Compositions Made from MEK

To a dram vial, wet silver saccharinate (1.64 g, 72% w/w solids in wet cake) was added followed by 20% w/w Avalure AC315 polymer solution (17.36 g) made in methyl ethyl ketone (MEK) solvent. The two ingredients were mixed to homogeneity. To this mixture, adhesive S390 (1 g) was added and again mixed in thoroughly to form an antimicrobial composition.

Adhesive composition was made by mixing 20% w/w Avalure AC315 polymer solution made in methyl ethyl ketone (MEK) solvent with adhesive S390 in 2 to 1 weight ratio.

The silver antimicrobial composition and adhesive composition were coated on brown paper and #42CK paper liners respectively using Meyer rods #40 and #20. They were dried at 110° C. for 3 minutes (adhesive composition) and 2 minutes (silver antimicrobial composition) respectively to form adhesive layer and an antimicrobial layer, and laminated similar to the samples in Example 23. The laminate constructs showed excellent differential release and in ZOI assay exhibited strong antimicrobial activity against MRSA.

Therefore, the laminate constructs made using MEK behaved the same way as those made with ethyl acetate.

### Example 33

### Laminate Construct with Tint for Improved Identification

The underlying EVA foam is white and the silver antimicrobial layer bonded to the foam is not always apparent. To distinguish EVA foam with silver antimicrobial layer, we prepared a laminate construct with a small amount of colorant added to the adhesive side to provide tint. Because the Avalure AC315 adhesive layer is transparent, the tint would show through.

The laminate construct with tint was prepared as follows: Approximately 5 mg of methylene blue dye was dissolved in 2 g of 20% w/w Avalure AC315 polymer solution made in MEK (note not all dye dissolved as few crystals were seen at the bottom of the test tube). To this dye-polymer solution, adhesive S390 solution (1 g) was added and the entire mixture vortexed to uniformity.

Using Meyer rod #20, the adhesive solution with methylene blue was coated on #42 CK paper liner (1″×4.5″) and dried at 110° C. for 3 minutes. The adhesive side was laminated to silver antimicrobial layer on a brown paper liner (1″×4.5″) from Example 25. The laminate was bonded to EVA foam strip, the brown liner removed to expose silver antimicrobial layer. The blue color tint was visible through the silver film.

### Example 34

### Discoloration Resistance Testing of Laminate Constructs

Silver saccharinate slurry was made similar to the composition of slurry in Example 25 except the amount of silver saccharinate was 6% w/w. Nineteen parts of the slurry was mixed with one part of S390 adhesive solution to obtain silver antimicrobial composition of roughly 45 kg. The silver antimicrobial composition was applied on brown paper liner (as in earlier examples) on a pilot coater resulting in ˜25 microns thick film after drying. Several pieces of silver antimicrobial layer brown paper liner were in 1″×1.5″ size and bonded with adhesive coating coated on #42 CK liner paper pieces to make laminate construct samples. These samples were attached to EVA foam pieces to obtain foam with silver antimicrobial layer. The samples were prepared as follows: 7 foam with silver antimicrobial layer strips were wrapped with red plastic film wrap (acetate gift wrap paper with thickness ˜1-1.5 mils) such that the wrapped portion covered half of the strip area. Similarly 7 foam with silver antimicrobial layer strips were wrapped in a blue film wrap of polyethylene.
Discoloration resistance testing was carried out as followed:
   1. One foam with silver antimicrobial layer strip each with partly covered with red and blue liners was stored in a desk drawer away from light throughout the test (Control samples).
   2. 3 foam with silver antimicrobial layer strips each with partly covered with red and blue liners were placed about ˜1′ under a table top desk incandescent lamp (60 W) for a period of at least 30 days recording any changes every week.
   3. The last set of foam with silver antimicrobial layer strips (3 of each kind) were exposed to direct sunlight for a total of 45 h (actual sunlight exposure) and changes at the end of the test duration were recorded.
The test results are summarized below:
   a. No discoloration was observed on any samples (3 of 3) exposed to table top desk lamp light. The regions under the red and blue colored films were no different from the exposed region. The silver antimicrobial layer of laminate constructs have excellent discoloration resistance to office light conditions. This property indicates that clear film packaging may be used for packaging devices having silver antimicrobial layer containing laminate constructs.
   b. No discoloration was observed on any samples (3 of 3) exposed to direct sunlight. The exposed region and the regions covered by red and blue films looked the same Therefore, the silver film laminate constructs of the present invention also possess excellent short term resistance to direct sunlight.

What is claimed is:

1. A laminate construct comprising at least one antimicrobial layer comprising at least one antimicrobial agent and a binder, and a second layer.

2. The laminate construct of claim 1, wherein an antimicrobial layer is contacting substantially all of the surface of one side of the second layer.

3. The laminate construct of claim 1, wherein the antimicrobial layer comprises at least one antimicrobial agent comprising, an antibiotic, antiseptic, silver, silver salts, silver nanoparticles, ionic silver, combinations of one or more one silver compounds, zinc, copper, gold, and their salts, quaternary ammonium salts, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, chlorohexidine, polyhexamethylene biguanides, polyhexamethylene biguanides, triclosan, iodine, iodine-polyvinyl pyrrolidone complex, urea-peroxide complex, benzalkonium salts, turmeric extract, natural anti-infective compounds, or combinations thereof.

4. The laminate construct of claim 1, wherein the antimicrobial layer further comprises one or more additives.

5. The laminate construct of claim 4, wherein an additive comprises colorants, food colors, one or more types of fluorescent compounds, fillers, titania, natural or synthetic clays, humectants, glycerol, urea, glycols, polyethylene glycol, or plasticizers.

6. The laminate construct of claim 1, wherein the second layer comprises an adhesive layer.

7. The laminate construct of claim 6, wherein the adhesive layer comprises a pressure sensitive adhesive, a permanent adhesive, a light-activated adhesive or heat-activated adhesive, a natural polymer adhesive, or a synthetic polymer adhesive, a cross-linked polymeric adhesive, or a noncross-linked polymeric adhesive.

8. The laminate construct of claim 7, wherein the adhesive polymer is polyurethane, silicone, casein, acrylic, polyisobutylene, polyacrylate, or stryrene.

9. The laminate construct of claim 1, further comprising at least one structural element.

10. The laminate construct of claim 9, wherein the structural element is a matrix material on which the antimicrobial layer has been formed.

11. The laminate construct of claim 10, wherein the matrix material is a woven or nonwoven material.

12. The laminate construct of claim 9, wherein the structural element is a liner.

13. The laminate construct of claim 12, wherein there are two structural elements, each of which is a liner.

14. The laminate construct of claim 13, wherein the two liners are the same material.

15. The laminate construct of claim 13, wherein the two liners are different materials.

16. The laminate construct of claim 13, wherein an antimicrobial layer is contacting one liner, and an adhesive layer is contacting the second liner.

17. A method of making a laminate construct comprising,
a. Applying an antimicrobial composition to a structural element to form a coating on the structural element;
b. Removing at least a portion of one or more solvents from the antimicrobial composition to form an antimicrobial layer;
c. Applying an adhesive composition to the outer surface of the antimicrobial layer;
d. Removing at least a portion of one or more solvents from the adhesive composition to form an adhesive layer; and
e. Optionally, adding a second structural element to cover the adhesive layer.

18. The method of claim 17, wherein the antimicrobial layer comprises at least one antimicrobial agent comprising, an antibiotic, antiseptic, silver, silver salts, silver nanoparticles, ionic silver, combinations of one or more one silver compounds, zinc, copper, gold, and their salts, quaternary ammonium salts, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, chlorohexidine, polyhexamethylene biguanides, polyhexamethylene biguanides, triclosan, iodine, iodine-polyvinyl pyrrolidone complex, urea-peroxide complex, benzalkonium salts, turmeric extract, natural anti-infective compounds, or combinations thereof.

19. The method of claim 17, wherein the antimicrobial layer further comprises one or more additives.

20. An article comprising a laminate construct of claim 1.
